(19) United States
(12) Patent Application Publication
(10) Pub. No.: US 2024/0197956 A1
(43) Pub. Date: Jun. 20, 2024

(54) METAL ALLOY HAVING ZIRCONIUM CONTAINING COATING

(71) Applicant: MiRus LLC, Marietta, GA (US)

(72) Inventors: Jordan Bauman, Smyrna, GA (US); Noah Roth, Marietta, GA (US); Jay Yadav, Atlanta, GA (US)

(21) Appl. No.: 18/417,939

(22) Filed: Jan. 19, 2024

Related U.S. Application Data
(63) Continuation-in-part of application No. 18/400,338, filed on Dec. 29, 2023.
(63) Continuation-in-part of application No. 18/204,180, filed on May 31, 2023.
(60) Provisional application No. 63/439,892, filed on Jan. 19, 2023.
(60) Provisional application No. 63/540,266, filed on Sep. 25, 2023.
(60) Provisional application No. 63/389,281, filed on Jul. 14, 2022.
(60) Provisional application No. 63/347,337, filed on May 31, 2022.

(51) Int. Cl. A61L 27/30; A61F 2/24

(52) U.S. Cl. CPC A61L 27/306; A61F 2/2418; A61F 2240/001; A61L 2300/404; A61L 2420/02; A61L 2430/20

(57) ABSTRACT

A medical device that includes an coating; and wherein the coating includes a) zirconium salt, b) zirconia, and/or c) Zr(IV) complexes.

### REFERENCED APPLICATIONS

The present disclosure claims priority on United States Provisional Application Serial No. Ser. No. 63/439,892, filed Jan. 19, 2023, which is fully incorporated herein.

The present application is a continuation-in-part of U.S. application Ser. No. 18/400,338 filed Dec. 29, 2023, which in turn priority claims priority to U.S. Provisional Application Ser. No. 63/540,266 filed Sep. 25, 2023, which are all fully incorporated herein by reference.

The present application is a continuation-in-part of U.S. application Ser. No. 18/204,180 filed May 31, 2023, which claims priority on U.S. Provisional Application Ser. No. 63/389,281 filed Jul. 14, 2022, which are all fully incorporated herein by reference.

The present application is a continuation-in-part of U.S. application Ser. No. 18/204,180 filed May 31, 2023, which claims priority on U.S. Provisional Application Ser. No. 63/347,337 filed May 31, 2022, which are all fully incorporated herein by reference.

### FIELD OF DISCLOSURE

The disclosure relates medical devices that are coated, particularly to medical devices that are coated with a zirconium containing coating, more particularly to medical devices that are coated with a zirconium containing coating to inhibit or prevent microbial growth and/or contamination of the outer surface of one or more portions of the medical device.

### BACKGROUND OF DISCLOSURE

During the final processing and storing of a medical device, the medical device is commonly disinfected to eliminate any microorganisms on the medical device. Thereafter, the medical device can optionally be coated with an antimicrobial material prior to packaging to inhibit or prevent microorganism growth on the medical device. Additionally or alternatively, prior to use, the medical device can be disinfected with an antimicrobial material. One common antimicrobial material that is used to disinfect medical devices is glutaraldehyde. Glutaraldehyde is commonly used when the medical device cannot be disinfected by use of heat (e.g., heart valves, etc.). Although glutaraldehyde is an effective disinfecting material, the use of glutaraldehyde is a volatile material that can be inhaled by both patients and medical personnel, thereby potentially causing irritation to the nasal, throat and/or lungs of the individuals that inhale the glutaraldehyde. Prolonged exposure to glutaraldehyde has been linked to other adverse health effects.

In view of the current state of the art of disinfecting medical devices, there is a need for an improved disinfecting material that a) has a lower volatility than glutaraldehyde, and b) that can be effectively applied to a variety of medical devices to effectively disinfect the medical device without the need to expose the medical device to a high temperature environment (e.g., 38+° C.; 38+° C. to 100+° C. and all values and ranges therebetween; 38+° C. to 150+° C. and all values and ranges therebetween).

### SUMMARY OF THE DISCLOSURE

The present disclosure is direct to zirconium metal and/or a zirconium containing coating that can be applied to a medical device to improved ability to identify the medical device, to improve the lubricity of the medical device, improve the biocompatibility of the medical device, and/or alter the antimicrobial behavior of the medical device, inhibit or prevent microbial growth and/or contamination of the outer surface of one or more portions of the medical device, create a smooth surface on the medical device (e.g., reduces micro-cracks or pores in the medical device), improve corrosion resistance of the medical device, improve wear resistance of the medical device, color code the medical device, improves the success rate of the implanted medical device, and/or form improved anti-galling surfaces. The present disclosure is direct to a medical device that is coated with a material, and wherein the coated orthopedic medical device has improved success rates after implantation. The medical device has one or more properties and/or features, namely a) is formed of a material that is fully absent nickel and/or chromium content or has reduced amounts of nickel and/or chromium as compared to medical devices that are partially or fully formed of standard stainless steel alloy, standard cobalt-chromium alloy, and standard TiNi alloy so as to reduce or eliminate problems associated with allergic reactions with surrounding tissue that can be at least partially caused by chromium and/or nickel, b) has reduced ion release of nickel and/or chromium so as to reduce or eliminate problems associated with allergic reactions with surrounding tissue that can be at least partially caused by chromium and/or nickel, c) has an outer surface that is absent nickel and/or chromium and/or has reduced amounts of nickel and/or chromium as compared to medical devices that are partially or fully formed of standard stainless steel alloy, standard cobalt-chromium alloy, and standard TiNi alloy so as to reduce or eliminate problems associated with allergic reactions with surrounding tissue that can be at least partially caused by chromium and/or nickel, d) is partially or fully formed of a metal alloy that has increased strength, increased surface hardness, and/or increased ductility as compared to standard stainless steel alloy, standard cobalt-chromium alloy, standard TiNi alloy, and standard TiAlV alloy, e) has an outer surface that promotes bone healing about the implanted medical device, f) has an outer surface that reduces bacterial growth and/or bacterial infection about the implanted medical device, and/or f) has an outer surface that improves the biocompatibility of the orthopedic medical device. The medical device can include an orthopedic device, PFO (patent foramen ovale) device, stent, valve (e.g., heart valve, TAVR valve, mitral valve replacement, tricuspid valve replacement, pulmonary valve replacement, etc.), spinal implant, frame and other structures for use with a spinal implant, vascular implant, graft, guide wire, sheath, catheter, needle, stent catheter, electrophysiology catheter, hypotube, staple, cutting device, any type of implant, pacemaker, dental implant, dental crown, dental braces, wire used in medical procedures, bone implant, artificial disk, artificial spinal disk, prosthetic implant or device to repair, replace and/or support a bone (e.g., acromion, atlas, axis, calcaneus, carpus, clavicle, coccyx, epicondyle, epitrochlea, femur, fibula, frontal bone, greater trochanter, humerus, ilium, ischium, mandible, maxilla, metacarpus, metatarsus, occipital bone, olecranon, parietal bone, patella, phalanx, radius, ribs, sacrum, scapula, sternum, talus, tarsus, temporal bone, tibia, ulna, zygomatic bone, etc.) and/or cartilage, bone plate, nail, rod, screw, post, cage, plate, pedicle screw, cap, hinge, joint system, anchor, spacer, shaft, anchor, disk, ball, tension band, locking connector other structural assembly that is used in a body to support a structure, mount a structure, and/or repair a structure in a body such as, but not limited to, a human body, animal body, etc. In one non-limiting embodiment, the medical device includes an expandable frame (e.g., stent, prosthetic heart valve, etc.) that can plastically deform radially outwardly by an expansion arrangement (e.g., inflatable balloon, etc.). One non-limiting heart valve is disclosed in U.S. Ser. No. 18/400,781 filed Dec. 29, 2023, which is fully incorporated herein by reference. In one non-limiting embodiment, the medical device is an orthopedic medical device is in the form of a spinal implant; frame and other structure for use with a spinal implant; bone implant; artificial disk; artificial spinal disk; spinal interbody; expandable spinal interbody; interbody fusion device; expandable interbody fusion device; prosthetic implant or device to repair, replace and/or support a bone (e.g., acromion, atlas, axis, calcaneus, carpus, clavicle, coccyx, epicondyle, epitrochlea, femur, fibula, frontal bone, greater trochanter, humerus, ilium, ischium, mandible, maxilla, metacarpus, metatarsus, occipital bone, olecranon, parietal bone, patella, phalanx, radius, ribs, sacrum, scapula, sternum, talus, tarsus, temporal bone, tibia, ulna, zygomatic bone, etc.) and/or cartilage; bone plate nail; spinal rod; bone screw; post; spinal cage; bone plate; pedicle screw; cap; hinge; joint system; anchor; spacer; shaft; anchor; disk; ball; tension band; locking connector or other structural assembly that is used in a body to support a structure, mount a structure, and/or repair a structure in a body such as, but not limited to, a human body, animal body, etc. In another non-limiting embodiment, the medical device is a spinal rod; pedicle screw; bone plate; artificial spinal disk; artificial spinal disk; spinal interbody; expandable spinal interbody; interbody fusion device; expandable interbody fusion device; or prosthetic implant or device to repair, replace and/or support a bone.

In accordance with another and/or alternative non-limiting aspect of the present disclosure, there is provided a medical device that is partially (e.g. 1-99.999 wt. % and all values and ranges therebetween) or fully formed of a metal material that includes a) standard stainless steel, b) standard CoCr alloy or standard MP35N alloy or a standard Phynox alloy or standard Elgiloy alloy or standard L605 alloy, c) standard TiAlV alloy, d) standard aluminum alloy, e) standard nickel alloy, f) standard titanium alloy, g) standard tungsten alloy, h) standard molybdenum alloy, i) standard copper alloy, j) standard beryllium-copper alloy, k) standard Nitinol alloy, 1) refractory metal alloy, or m) metal alloy that includes at least 5 atomic weight percent (awt. %) or atomic percent (awt %) rhenium (e.g., 5-99 awt. % rhenium and all values and ranges therebetween). As used herein, atomic weight percent (awt. %) or atomic percent (awt %) are used interchangeably. As defined herein, the weight percentage (wt. %) of an element is the weight of that element measured in the sample divided by the weight of all elements in the sample multiplied by 100. The atomic percentage or atomic weight percent (awt %) is the number of atoms of that element, at that weight percentage, divided by the total number of atoms in the sample multiplied by 100. The use of the terms weight percentage (wt. %) and atomic percentage or atomic weight percentage (awt. %) are two ways of referring to metallic alloy and its constituents. As defined herein, a standard stainless-steel alloy (SS alloy) includes 10-28 wt. % (weight percent) chromium, 0-35 wt. % nickel, 0-4 wt. % molybdenum, 0-2 wt. % manganese, 0-0.75 wt. % silicon, 0-0.3 wt. % carbon, 0-5 wt. % titanium, 0-10 wt. % niobium, 0-5 wt. % copper, 0-4 wt. % aluminum, 0-10 wt. % tantalum, 0-1 wt. % Se, 0-2 wt. % vanadium, 0-2 wt. % tungsten, and at least 50 wt. % iron. A standard 316L alloy that falls within a standard stainless-steel alloy includes 17-19 wt. % chromium, 13-15 wt. % nickel, 2-4 wt. % molybdenum, 2 wt. % max manganese, 0.75 wt. % max silicon, 0.03 wt. % max carbon, balance iron. As defined herein, a standard cobalt-chromium alloy (CoCr alloy) includes 15-32 wt. % chromium, 1-38 wt. % nickel, 2-18 wt. % molybdenum, 0-18 wt. % iron, 0-1 wt. % titanium, 0-0.15 wt. % manganese, 0-0.15 wt. % silver, 0-0.25 wt. % carbon, 0-16 wt. % tungsten, 0-2 wt. % silicon, 0-2 wt. % aluminum, 0-1 wt. % iron, 30-68 wt. % cobalt, 0-0.1 wt. % boron, 0-0.15 wt. % silver, and 0-2 wt. % titanium. As a standard MP35N alloy that falls within a standard CoCr alloy includes 18-22 wt. % chromium, 32-38 wt. % nickel, 8-12 wt. % molybdenum, 0-2 wt. % iron, 0-0.5 wt. % silicon, 0-0.5 wt. % manganese, 0-0.2 wt. % carbon, 0-2 wt. % titanium, 0-0.1 wt. %, 0-0.1 wt. % boron, 0-0.15 wt. % silver, and balance cobalt. As defined herein, a standard Phynox and standard Elgiloy alloy that falls within a standard CoCr alloy includes 38-42 wt. % cobalt, 18-22 wt. % chromium, 14-18 wt. % iron, 13-17 wt. % nickel, 6-8 wt. % molybdenum. As defined herein, a standard L605 alloy that falls within a standard CoCr alloy includes 18-22 wt. % chromium, 14-16 wt. % tungsten, 9-11 wt. % nickel, balance cobalt. As defined herein, a standard titanium-aluminum-vanadium alloy (TiAlV alloy) includes 5.5-6.75 wt. % aluminum, 3.5-4.5 wt. % vanadium, 85-93 wt. % titanium, 0-0.4 wt. % iron, 0-0.2 wt. % carbon. A standard Ti-6Al-4V alloy that falls with a standard TiAlV alloy includes incudes 3.5-4.5 wt. % vanadium, 5.5-6.75 wt. % aluminum, 0.3 wt. % max iron, 0.08 wt. % max carbon, 0.05 wt. % max yttrium, balance titanium. As defined herein, a standard aluminum alloy includes 80-99 wt. % aluminum, 0-12 wt. % silicon, 0-5 wt. % magnesium, 0-1 wt. % manganese, 0-0.5 wt. % scandium, 0-0.5 wt. % beryllium, 0-0.5 wt. % yttrium, 0-0.5 wt. % cerium, 0-0.5 wt. % chromium, 0-3 wt. % iron, 0-0.5, 0-9 wt. % zinc, 0-0.5 wt. % titanium, 0-3 wt. % lithium, 0-0.5 wt. % silver, 0-0.5 wt. % calcium, 0-0.5 wt. % zirconium, 0-1 wt. % lead, 0-0.5 wt. % cadmium, 0-0.05 wt. % bismuth, 0-1 wt. % nickel, 0-0.2 wt. % vanadium, 0-0.1 wt. % gallium, and 0-7 wt. % copper. As defined herein, a standard nickel alloy includes 30-98 wt. % nickel, 5-25 wt. % chromium, 0-65 wt. % iron, 0-30 wt. % molybdenum, 0-32 wt. % copper, 0-32 wt. % cobalt, 2-2 wt. % aluminum, 0-6 wt. % tantalum, 0-15 wt. % tungsten, 0-5 wt. % titanium, 0-6 wt. % niobium, 0-3 wt. % silicon. As defined herein, a standard titanium alloy includes 80-99 wt. % titanium, 0-6 wt. % aluminum, 0-3 wt. % tin, 0-1 wt. % palladium, 0-8 wt. % vanadium, 0-15 wt. % molybdenum, 0-1 wt. % nickel, 0-0.3 wt. % ruthenium, 0-6 wt. % chromium, 0-4 wt. % zirconium, 0-4 wt. % niobium, 0-1 wt. % silicon, 0.0.5 wt. % cobalt, 0-2 wt. % iron. As defined herein, a standard tungsten alloy includes 85-98 wt. % tungsten, 0-8 wt. % nickel, 0-5 wt. % copper, 0-5 wt. % molybdenum, 0-4 wt. % iron. As defined herein, a standard molybdenum alloy includes 90-99.5 wt. % molybdenum, 0-1 wt. % nickel, 0-1 wt. % titanium, 0-1 wt. % zirconium, 0-30 wt. % tungsten, 0-2 wt. % hafnium, 0-2 wt. % lanthanum. As defined herein, a standard copper alloy includes 55-95 wt. % copper, 0-40 wt. % zinc, 0-10 wt. % tin, 0-10 wt. % lead, 0-1 wt. % iron, 0-5 wt. % silicon, 0-12 wt. % manganese, 0-12 wt. % aluminum, 0-3 wt. % beryllium, 0-1 wt. % cobalt, 0-20 wt. % nickel. As defined herein, a standard beryllium-copper alloy includes 95-98.5 wt. % copper, 1-4 wt. % beryllium, 0-1 wt. % cobalt, and 0-0.5 wt. % silicon. As defined herein, a standard titanium-nickel alloy (e.g., Nitinol alloy) includes 42-58 wt. % nickel and 42-58 wt. % titanium. As defined herein, a refractory metal alloy is a metal alloy that includes at least 20 wt. % of one or more of molybdenum, rhenium, niobium, tantalum or tungsten. Non-limiting refractory metal alloys include MoRe alloy, ReW alloy, MoReCr alloy, MoReTa alloy, MoReTi alloy, WCu alloy, ReCr, molybdenum alloy, rhenium alloy, tungsten alloy, tantalum alloy, niobium alloy, etc.

In accordance with another and/or alternative non-limiting aspect of the present disclosure, the medical device is partially or fully formed of a metal material that includes a metal alloy that contains at least 15 awt. % rhenium. It has been found that for several metal alloys the inclusion of at least 15 awt % rhenium results in the ductility and/or tensile strength of the metal alloy to improve as compared to a metal alloy is that absent rhenium. Such improvement in ductility and/or tensile strength due to the inclusion of at least 15 awt. % rhenium in the metal alloy is referred to as the “rhenium effect.” As defined herein, a “rhenium effect” is a) an increase of at least 10% in ductility of the metal alloy caused by the addition of rhenium to the metal alloy, and/or b) an increase of at least 10% in tensile strength of the metal alloy caused by the addition of rhenium to the metal alloy. It has been found for some metal alloys (e.g., standard stainless steel, standard CoCr alloys, standard TiAlV alloys, standard aluminum alloys, standard nickel alloys, standard titanium alloys, standard tungsten alloys, standard molybdenum alloys, standard copper alloys, standard MP35N alloys, standard beryllium-copper alloys, etc.), the inclusion of at least 15 awt. % rhenium results in improved ductility and/or tensile strength. It has been found that the addition of rhenium to a metal alloy can result in the formation of a twining alloy in the metal alloy that results in the overall ductility of the metal alloy to increase as the yield and tensile strength increases as a result of reduction and/or work hardening of the metal alloy that includes the rhenium addition. The rhenium effect has been found to occur when the atomic weight of rhenium in the metal alloy is at least 15% (e.g., 15-99 awt. % rhenium in the metal alloy and all values and ranges therebetween). For example, for standard stainless-steel alloys, the rhenium effect can begin to be present when the stainless-steel alloy is modified to include a rhenium amount of at least 5-10 wt. % (and all values and ranges therebetween) of the stainless-steel alloy. For standard CoCr alloys, the rhenium effect can begin to be present when the CoCr alloy is modified to include a rhenium amount of at least 4.8-9.5 wt. % (and all values and ranges therebetween) of the CoCr alloy. For standard TiAlV alloys, the rhenium effect can begin to be present when the TiAlV alloy is modified to include a rhenium amount of at least 4.5-9 wt. % (and all values and ranges therebetween) of the TiAlV alloy. It can be appreciated, the rhenium content in the above non-limiting examples can be greater than the minimum amount to create the rhenium effect in the metal alloy.

In accordance with another and/or alternative aspect of the present disclosure, the metal alloy includes at least 5 awt. % (e.g., 5-99 awt. % and all values and ranges therebetween) rhenium, and at least 0.1 wt. % (e.g., 0.1 wt. % to 96 wt. % and all values and ranges therebetween) of one or more of aluminum, boron, beryllium, bismuth, cadmium, calcium, cerium, cerium oxide, chromium, cobalt, copper, gallium, gold, hafnium, iridium, iron, lanthanum, lanthanum oxide, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, yttrium oxide, zinc, zirconium, and/or zirconium oxide, and the metal alloy optionally includes 0-2 wt. % (and all values and ranges therebetween) of a combination of other metals, carbon, oxygen, phosphorous, sulfur, hydrogen and/or nitrogen, and which metal alloy exhibits a rhenium effect. In another non-limiting embodiment, the metal alloy that is used to partially or fully form the medical device is a refractory metal alloy. In one non-limiting embodiment, the metal alloy that is used to partially or fully form the medical device is a standard stainless-steel alloy that has been modified to include at least 15 awt. % rhenium. In one non-limiting embodiment, the metal alloy that is used to partially or fully form the medical device is a standard cobalt chromium alloy that has been modified to include at least 15 awt. % rhenium. In one non-limiting embodiment, the metal alloy that is used to partially or fully form the medical device is a standard TiAlV alloy that has been modified to include at least 15 awt. % rhenium. In one non-limiting embodiment, the metal alloy that is used to partially or fully form the medical device is a standard aluminum alloy that has been modified to include at least 15 awt. % rhenium. In one non-limiting embodiment, the metal alloy that is used to partially or fully form the medical device is a standard nickel alloy that has been modified to include at least 15 awt. % rhenium. In one non-limiting embodiment, the metal alloy that is used to partially or fully form the medical device is a standard titanium alloy that has been modified to include at least 15 awt. % rhenium. In one non-limiting embodiment, the metal alloy that is used to partially or fully form the medical device is a standard tungsten alloy that has been modified to include at least 15 awt. % rhenium. In one non-limiting embodiment, the metal alloy that is used to partially or fully form the medical device is a standard molybdenum alloy that has been modified to include at least 15 awt. % rhenium. In one non-limiting embodiment, the metal alloy that is used to partially or fully form the medical device is a standard copper alloy that has been modified to include at least 15 awt. % rhenium. In one non-limiting embodiment, the metal alloy that is used to partially or fully form the medical device is a standard beryllium-copper alloy that has been modified to include at least 15 awt. % rhenium.

In accordance with another and/or alternative aspect of the present disclosure, the metal alloy that is used to partially or fully form the medical device includes rhenium and molybdenum, and the weight percent of rhenium in the metal alloy is greater that the weight percent of molybdenum in the metal alloy. In one non-limiting embodiment, the metal alloy includes rhenium and molybdenum, and the weight percent of rhenium in the metal alloy is greater that the weight percent of molybdenum in the metal alloy, and the weight percent of one or more of bismuth, niobium, tantalum, tungsten, titanium, vanadium, chromium, manganese, yttrium, zirconium, technetium, ruthenium, rhodium, hafnium, osmium, copper, and iridium in the metal alloy is greater that the weight percent of molybdenum in the metal alloy, and the metal alloy optionally includes 0-2 wt. % of a combination of other metals, carbon, oxygen, phosphorous, sulfur, hydrogen and/or nitrogen.

In accordance with another and/or alternative aspect of the present disclosure, the metal alloy that is used to partially or fully form the medical device includes rhenium and molybdenum, and the atomic weight percent of rhenium to the atomic weight percent of the combination of one or more of bismuth, niobium, tantalum, tungsten, titanium, vanadium, chromium, manganese, yttrium, zirconium, technetium, ruthenium, rhodium, hafnium, osmium, copper, and iridium is 0.4:1 to 2.5:1 (and all values and ranges therebetween).

In accordance with another and/or alternative aspect of the present disclosure, the metal alloy that is used to partially or fully form the medical device includes at least 5 awt. % (e.g., 5-99 awt. % and all values and ranges therebetween) rhenium plus at least two metals selected from the group of molybdenum, bismuth, chromium, iridium, niobium, tantalum, titanium, yttrium, and zirconium, and the content of the metal alloy that includes other elements and compounds is 0-0.1 wt. %. In another non-limiting embodiment, the metal alloy includes rhenium, molybdenum, and chromium. In another non-limiting embodiment, the metal alloy includes at least 35 wt. % (e.g., 35-75 wt. % and all values and ranges therebetween) rhenium, and the metal alloy also includes chromium. In one non-limiting embodiment, the metal alloy includes at least 35 wt. % rhenium and at least 25 wt. % (e.g., 25-49.9 wt. % and all values and ranges therebetween) of the metal alloy includes chromium, and optionally 0.1-40 wt. % (and all values and ranges therebetween) of the metal alloy includes one or more of aluminum, bismuth, calcium, carbon, cerium oxide, cobalt, copper, gold, hafnium, iridium, iron, lanthanum, lanthanum oxide, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, and/or zirconium oxide, and optionally 0-2 wt. % of a combination of other metals, carbon, oxygen, phosphorous, sulfur, hydrogen and/or nitrogen. In another non-limiting embodiment, the metal alloy includes 15-50 awt. % rhenium (and all values and ranges therebetween) and 0.5-70 awt. % chromium (and all values and ranges therebetween). In another non-limiting embodiment, the metal alloy includes 15-50 awt. % rhenium (and all values and ranges therebetween) and 0.5-70 awt. % tantalum (and all values and ranges therebetween). In another non-limiting embodiment, the metal alloy includes 15-50 awt. % rhenium (and all values and ranges therebetween) and 0.5-70 awt. % niobium (and all values and ranges therebetween). In another non-limiting embodiment, the metal alloy includes 15-50 awt. % rhenium (and all values and ranges therebetween) and 0.5-70 awt. % titanium (and all values and ranges therebetween). In another non-limiting embodiment, the metal alloy includes 15-50 awt. % rhenium (and all values and ranges therebetween) and 0.5-70 awt. % zirconium (and all values and ranges therebetween). In another non-limiting embodiment, the metal alloy includes 15-50 awt. % rhenium (and all values and ranges therebetween) and 0.5-70 awt. % molybdenum (and all values and ranges therebetween). In another non-limiting embodiment, the metal alloy includes at least 15 awt. % rhenium, greater than 50 wt. % titanium (e.g., 51-80 wt. % and all values and ranges therebetween), 15-45 wt. % (and all values and ranges therebetween) niobium, 0-10 wt. % (and all values and ranges therebetween) zirconium, 0-15 wt. % (and all values and ranges therebetween) tantalum, and 0-8 wt. % molybdenum (and all values and ranges therebetween).

In accordance with another and/or alternative aspect of the present disclosure, the metal alloy that is used to partially or fully form the medical device includes at least 0.1 wt. % (e.g., 0.1-70 wt. % and all values and ranges therebetween) rhenium and one or more metals selected from the group of molybdenum, chromium, cobalt, nickel, titanium, tantalum, niobium, zirconium, and/or tungsten. In one non-limiting embodiment, the metal alloy that is used to partially or fully form the medical device includes at least 5 wt. % (e.g., 5-70 wt. % and all values and ranges therebetween) rhenium and one or more metals selected from the group of molybdenum, chromium, cobalt, nickel, titanium, tantalum, niobium, zirconium, and/or tungsten.

Several non-limiting examples of metal alloys that can be used to partially or fully form the medical device are set forth below in weight percent:

| Component/Wt. % | Ex. 1 | Ex. 2 | Ex. 3 | Ex. |
|---|---|---|---|---|
| Al | 0-35% | 0-30% | 0-25% | 0-10% |
| Bi | 0-20% | 0-20% | 0-20% | 0-20% |
| Cr | 0-60% | 0-35% | 0-30% | 0-25% |
| Co | 0-60% | 0-50% | 0-40% | 0-20% |
| Mo | 0-95% | 0-80% | 0-55% | 0-30% |
| Nb | 0-80% | 0-60% | 0-50% | 0-20% |
| Ni | 0-60% | 0-55% | 0-40% | 0-20% |
| Re | 0.1-70% | 4.5-70% | 5-70% | 5-70% |
| Ta | 0-80% | 0-50% | 0-40% | 0-25% |
| Ti | 0-60% | 0-55% | 0-40% | 0-20% |
| V | 0-20% | 0-15% | 0-10% | 0-10% |
| W | 0-80% | 0-70% | 0-50% | 0-20% |
| Y | 0-20% | 0-15% | 0-10% | 0-10% |
| Zr | 0-20% | 0-15% | 0-10% | 0-10% |
| Component/Wt. % | Ex. 5 | Ex. 6 | Ex. 7 | Ex. 8 |
| Ag | 0-20% | 0-20% | 0-20% | 0-20% |
| Al | 0-35% | 0-30% | 5-30% | 0-25% |
| Bi | 0-20% | 0-20% | 0-20% | 0-20% |
| Cr | 10-40% | 0-40% | 0-40% | 0-40% |
| Cu | 0-20% | 0-20% | 0-20% | 0-20% |
| Co | 10-60% | 0-60% | 0-60% | 0-60% |
| Fe | 0-80% | 30-80% | 0-80% | 0-70% |
| Hf | 0-20% | 0-20% | 0-20% | 0-20% |
| Ir | 0-20% | 0-20% | 0-20% | 0-20% |
| Mg | 0-20% | 0-20% | 0-20% | 0-20% |
| Mn | 0-20% | 0-40% | 0-20% | 0-20% |
| Mo | 0-60% | 0-60% | 0-80% | 0-70% |
| Nb | 0-60% | 0-60% | 0-65% | 20-60% |
| Ni | 0-60% | 5-55% | 0-52% | 0-50% |
| Os | 0-20% | 0-20% | 0-20% | 0-20% |
| Pt | 0-20% | 0-20% | 0-20% | 0-20% |
| Re | 4.5-98% | 4.5-90% | 4.5-80% | 4.5-70% |
| Rh | 0-20% | 0-20% | 0-20% | 0-20% |
| Si | 0-20% | 0-20% | 0-20% | 0-20% |
| Sn | 0-20% | 0-20% | 0-20% | 0-20% |
| Ta | 0-60% | 0-60% | 5-65% | 0-60% |
| Tc | 0-20% | 0-20% | 0-20% | 0-20% |
| Ti | 0-60% | 0-55% | 0-53% | 0-50% |
| V | 0-20% | 0-20% | 2-20% | 0-20% |
| W | 0-60% | 0-60% | 0-80% | 0-70% |
| Y | 0-20% | 0-20% | 0-20% | 0-20% |
| Zr | 0-20% | 0-20% | 0-20% | 5-20% |
| Component/Wt. % | Ex. 9 | Ex. 10 | Ex. 11 | Ex. 12 |
| Ag | 0-5% | 0-5% | 0-5% | 0-5% |
| Al | 0-5% | 0-5% | 1-15% | 0-20% |
| Bi | 0-5% | 0-5% | 0-5% | 0-5% |
| Cr | 1-28% | 1-30% | 0-5% | 0-30% |
| Cu | 0-20% | 0-5% | 0-5% | 0-25% |
| Co | 0-5% | 1-60% | 0-5% | 0-60% |
| Fe | 10-80% | 0-25% | 0-5% | 0-80% |
| Hf | 0-5% | 0-5% | 0-5% | 0-5% |
| Ir | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-8% | 0-25% | 0-5% | 0-98% |
| Nb | 0-5% | 0-5% | 0-5% | 0-95% |
| Ni | 1-20% | 1-45% | 0-5% | 0-50% |
| Os | 0-5% | 0-5% | 0-5% | 0-5% |
| Pt | 0-5% | 0-5% | 0-5% | 0-5% |
| Re | 5-20% | 4.8-20% | 4.5-20% | 4.5-20% |
| Rh | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 0-5% | 0-98% |
| Tc | 0-5% | 0-5% | 0-5% | 0-5% |
| Ti | 0-5% | 0-5% | 40-93% | 0-93% |
| V | 0-5% | 0-5% | 1-10% | 0-20% |
| W | 0-5% | 0-20% | 0-5% | 0-98% |
| Y | 0-5% | 0-5% | 0-5% | 0-5% |
| Zr | 0-5% | 0-5% | 0-5% | 0-5% |
| Component/Wt. % | Ex. 13 | Ex. 14 | Ex. 15 | Ex. 16 |
| Mo | 40-80% | 40-80% | 40-80% | 40-80% |
| Co | ≤0.002% | ≤0.002% | ≤0.002% | ≤0.002% |
| Fe | ≤0.02% | ≤0.02% | ≤0.02% | ≤0.02% |
| Hf | 0.1-2.5% | 0-2.5% | 0-2.5% | 0-2.5% |
| Os | ≤1% | ≤1% | ≤1% | ≤1% |
| Nb | ≤0.01% | ≤0.01% | ≤0.01% | ≤0.01% |
| Pt | ≤1% | ≤1% | ≤1% | ≤1% |
| Re | 7-49% | 7.5-49% | 7.5-49% | 7.5-49% |
| Sn | ≤0.002% | ≤0.002% | ≤0.002% | ≤0.002% |
| Ta | 0-50% | 0-50% | 0-50% | 0-50% |
| Tc | ≤1% | ≤1% | ≤1% | ≤1% |
| Ti | ≤1% | ≤1% | ≤1% | ≤1% |
| V | ≤1% | ≤1% | ≤1% | ≤1% |
| W | 0-50% | 0-50% | 0-50% | 0.5-50% |
| Zr | ≤1% | ≤1% | ≤1% | ≤1% |
| Component/Wt. % | Ex. 17 | Ex. 18 | Ex. 19 | |
| Mo | 40-80% | 40-80% | 40-80% | |
| Co | ≤0.002% | ≤0.002% | ≤0.002% | |
| Hf | 0-2.5% | 0-2.5% | 0-2.5% | |
| Os | ≤1% | ≤1% | ≤1% | |
| Nb | ≤0.01% | ≤0.01% | ≤0.01% | |
| Pt | ≤1% | ≤1% | ≤1% | |
| Re | 7-49% | 7.5-49% | 7.5-49% | |
| Sn | ≤0.002% | ≤0.002% | ≤0.002% | |
| Ta | 0-50% | 0.5-50% | 0-50% | |
| Tc | ≤1% | ≤1% | ≤1% | |
| Ti | ≤1% | ≤1% | ≤1% | |
| V | ≤1% | ≤1% | ≤1% | |
| W | 0-50% | 0-50% | 0-50% | |
| Component/Wt. % | Ex. 20 | Ex. 21 | Ex. 22 | |
| Mo | 45-78% | 45-75% | 45-70% | |
| Co | <0.002% | ≤0.002% | ≤0.002% | |
| Hf | 0-2.5% | 0-2.5% | 0-2.5% | |
| Os | ≤1% | ≤1% | ≤1% | |
| Nb | ≤0.01% | ≤0.01% | ≤0.01% | |
| Pt | ≤1% | ≤1% | ≤1% | |
| Re | 7-49% | 7.5-49% | 7.5-49% | |
| Sn | ≤0.002% | ≤0.002% | ≤0.002% | |
| Ta | 0-50% | 0.5-50% | 0-50% | |
| Tc | ≤1% | ≤1% | ≤1% | |
| Ti | ≤1% | ≤1% | ≤1% | |
| V | ≤1% | ≤1% | ≤1% | |
| W | 0-50% | 0-50% | 0-50% | |
| Component/Wt. % | Ex. 23 | Ex. 24 | Ex. 25 | Ex. 26 |
| Mo | 35-80% | 35-80% | 35-70% | 35-65% |
| C | 0.05-0.15% | 0-0.15% | 0-0.15% | 0-0.15% |
| Hf | 0.8-1.4% | 0-2% | 0-2.5% | 0-2.5% |
| Re | 7-49% | 7-49% | 7.5-49% | 7.5-49% |
| Ta | 0-2% | 0-2% | 0-50% | 0-50% |
| W | 0-2% | 0-2% | 0-50% | 20-50% |
| Component/Wt. % | Ex. 27 | Ex. 28 | Ex. 29 | |
| Mo | 40-60% | 35-60% | 30-60% | |
| Hf | 0-2.5% | 0-2.5% | 0-2.5% | |
| Re | 7-60% | 7.5-65% | 7.5-70% | |
| Ta | 0-3% | 10-50% | 0-40% | |
| W | 0-3% | 0-50% | 0-40% | |
| Component/Wt. % | Ex. 30 | Ex. 31 | Ex. 32 | |
| W | 20-80% | 60-80% | 20-78% | |
| Re | 7.5-47.5% | 10-40% | 8-47.5% | |
| Mo | 0-47.5% | <0.5% | 1-47.5% | |
| Cu | <0.5% | <0.5% | <0.5% | |
| Co | ≤0.002% | ≤0.002% | ≤0.002% | |
| Fe | ≤0.02% | ≤0.02% | ≤0.02% | |
| Hf | <0.5% | <0.5% | <0.5% | |
| Os | <0.5% | <0.5% | <0.5% | |
| Nb | ≤0.01% | ≤0.01% | ≤0.01% | |
| Pt | <0.5% | <0.5% | <0.5% | |
| Sn | ≤0.002% | ≤0.002% | ≤0.002% | |
| Ta | <0.5% | <0.5% | <0.5% | |
| Tc | <0.5% | <0.5% | <0.5% | |
| Ti | <0.5% | <0.5% | <0.5% | |
| V | <0.5% | <0.5% | <0.5% | |
| Zr | <0.5% | <0.5% | <0.5% | |
| Component/Wt. % | Ex. 33 | Ex. 34 | Ex. 35 | |
| W | 20-80% | 60-80% | 20-75% | |
| Re | 7.5-47.5% | 10-40% | 7.5-47.5% | |
| Mo | 0-47.5% | <0.5% | 1-47.5% | |
| Component/Wt. % | Ex. 36 | Ex. 37 | Ex. 38 | |
| W | 50.1-80% | 65-80% | 50.1-79% | |
| Re | 10-40% | 10-35% | 10-40% | |
| Mo | 0-40% | <0.5% | 1-30% | |
| Component/Wt. % | Ex. 39 | Ex. 40 | Ex. 41 | |
| W | 20-49% | 20-49% | 20-49% | |
| Re | 7.5-60% | 7.5-60% | 7.5-60% | |
| Mo | 0-40% | 0-40% | 0-39% | |
| Component/Wt. % | Ex. 42 | Ex. 43 | Ex. 44 | |
| Re | 5-98% | 60-95% | 80-90% | |
| Mo | 0-80% | 0-40% | 0-20% | |
| W | 0-80% | 0-40% | 0-20% | |
| Component/Wt. % | Ex. 45 | Ex. 46 | Ex. 47 | |
| W | 20-49% | 20-49% | 20-49% | |
| Re | 6-40% | 6-40% | 6-39% | |
| Mo | 20-60% | 30-60% | 40-60% | |
| Component/Wt. % | Ex. 48 | Ex. 49 | Ex. 50 | |
| W | 20-40% | 20-35% | 20-30% | |
| Re | 6-40% | 6-40% | 6-40% | |
| Mo | 0-40% | 10-40% | 31-40% | |
| Component/Wt. % | Ex. 51 | Ex. 52 | Ex. 53 | Ex. 54 |
| Re | 5-60% | 5-60% | 5-60% | 5-60% |
| Mo | 0-55% | 10-55% | 10-55% | 10-55% |
| Bi | 1-42 | 0-32 | 0-32 | 0-32 |
| Cr | 0-32 | 1-42 | 0-32 | 0-32 |
| Ir | 0-32 | 0-32 | 1-42 | 0-32 |
| Nb | 0-32 | 0-32 | 0-32 | 1-42 |
| Ta | 0-32 | 0-32 | 0-32 | 0-32 |
| Ti | 0-32 | 0-32 | 0-32 | 0-32 |
| Y | 0-32 | 0-32 | 0-32 | 0-32 |
| Zr | 0-32 | 0-32 | 0-32 | 0-32 |
| Component/Wt. % | Ex. 55 | Ex. 56 | Ex. 57 | Ex. 58 |
| Re | 5-60% | 5-60% | 5-60% | 5-60% |
| Mo | 15-55% | 15-55% | 15-55% | 15-55% |
| Bi | 0-32 | 0-32 | 0-32 | 0-32 |
| Cr | 0-32 | 0-32 | 0-32 | 0-32 |
| Ir | 0-32 | 0-32 | 0-32 | 0-32 |
| Nb | 0-32 | 0-32 | 0-32 | 0-32 |
| Ta | 1-42 | 0-32 | 0-32 | 0-32 |
| Ti | 0-32 | 1-42 | 0-32 | 0-32 |
| Y | 0-32 | 0-32 | 1-42 | 0-32 |
| Zr | 0-32 | 0-32 | 0-32 | 1-42 |
| Component/Wt. % | Ex. 59 | Ex. 60 | Ex. 61 | Ex. 62 |
| Re | 41-59% | 41-59% | 41-59% | 41-59% |
| Mo | 18-45% | 18-45% | 18-45% | 18-45% |
| Bi | 1-42 | 0-32 | 0-32 | 0-32 |
| Cr | 0-32 | 1-42 | 0-32 | 0-32 |
| Ir | 0-32 | 0-32 | 1-42 | 0-32 |
| Nb | 0-32 | 0-32 | 0-32 | 1-42 |
| Ta | 0-32 | 0-32 | 0-32 | 0-32 |
| Ti | 0-32 | 0-32 | 0-32 | 0-32 |
| Y | 0-32 | 0-32 | 0-32 | 0-32 |
| Zr | 0-32 | 0-32 | 0-32 | 0-32 |
| Component/Wt. % | Ex. 63 | Ex. 64 | Ex. 65 | Ex. 66 |
| Re | 41-59% | 41-59% | 41-59% | 41-59% |
| Mo | 18-45% | 18-45% | 18-45% | 18-45% |
| Bi | 0-32 | 0-32 | 0-32 | 0-32 |
| Cr | 0-32 | 0-32 | 0-32 | 0-32 |
| Ir | 0-32 | 0-32 | 0-32 | 0-32 |
| Nb | 0-32 | 0-32 | 0-32 | 0-32 |
| Ta | 1-42 | 0-32 | 0-32 | 0-32 |
| Ti | 0-32 | 1-42 | 0-32 | 0-32 |
| Y | 0-32 | 0-32 | 1-42 | 0-32 |
| Zr | 0-32 | 0-32 | 0-32 | 1-42 |
| Component/Wt. % | Ex. 67 | Ex. 68 | Ex. 69 | Ex. 70 |
| Re | 41-59% | 41-59% | 41-59% | 41-59% |
| Mo | 18-45% | 18-45% | 18-45% | 18-45% |
| Bi | 0-15 | 0-15 | 1-36 | 0-15 |
| Cr | 1-20 | 1-20 | 1-20 | 1-20 |
| Ir | 0-15 | 0-15 | 0-15 | 0-15 |
| Nb | 1-36 | 0-15 | 0-15 | 0-15 |
| Ta | 0-15 | 1-36 | 0-15 | 0-15 |
| Ti | 0-15 | 0-15 | 0-15 | 0-15 |
| Y | 0-15 | 0-15 | 0-15 | 0-15 |
| Zr | 0-15 | 0-15 | 0-15 | 1-36 |
| Component/Wt. % | Ex. 71 | Ex. 72 | Ex. 73 | Ex. 74 |
| Re | 41-59% | 41-59% | 41-59% | 41-59% |
| Mo | 18-45% | 18-45% | 18-45% | 18-45% |
| Bi | 1-36 | 0-15 | 0-15 | 0-15 |
| Cr | 1-20 | 1-20 | 1-20 | 1-20 |
| Ir | 0-15 | 1-36 | 0-15 | 0-15 |
| Nb | 0-15 | 0-15 | 0-15 | 0-15 |
| Ta | 0-15 | 0-15 | 0-15 | 0-15 |
| Ti | 0-15 | 0-15 | 1-36 | 0-15 |
| Y | 0-15 | 0-15 | 0-15 | 1-36 |
| Zr | 0-15 | 0-15 | 0-15 | 0-15 |
| Component/Wt. % | Ex. 75 | Ex. 76 | Ex. 77 | Ex. 78 |
| Re | 41-59% | 41-59% | 41-59% | 41-59% |
| Mo | 18-45% | 18-45% | 18-45% | 18-45% |
| Bi | 1-34 | 0-15 | 0-15 | 0-15 |
| Cr | 0-15 | 0-15 | 0-15 | 0-15 |
| Ir | 0-15 | 0-15 | 0-15 | 1-34 |
| Nb | 3-27 | 3-27 | 3-27 | 3-27 |
| Ta | 0-42 | 1-34 | 0-15 | 0-15 |
| Ti | 0-15 | 0-15 | 0-15 | 0-15 |
| Y | 0-15 | 0-15 | 0-15 | 0-15 |
| Zr | 0-15 | 0-15 | 3-27 | 0-15 |
| Component/Wt. % | Ex. 79 | Ex. 80 | Ex. 81 | Ex. 82 |
| Re | 41-59% | 41-59% | 41-59% | 41-59% |
| Mo | 18-45% | 18-45% | 18-45% | 18-45% |
| Bi | 0-15 | 0-15 | 0-15 | 0-15 |
| Cr | 0-15 | 0-15 | 0-15 | 0-15 |
| Ir | 0-15 | 1-34 | 0-15 | 0-15 |
| Nb | 0-15 | 0-15 | 0-15 | 0-15 |
| Ta | 1-34 | 0-15 | 3-27 | 0-15 |
| Ti | 0-15 | 0-15 | 0-15 | 0-15 |
| Y | 0-15 | 0-15 | 0-15 | 3-27 |
| Zr | 3-27 | 3-27 | 3-27 | 3-27 |
| Component/Wt. % | Ex. 83 | Ex. 84 | Ex. 85 | Ex. 86 |
| Re | 41-59% | 41-59% | 41-59% | 41-59% |
| Mo | 18-45% | 18-45% | 18-45% | 18-45% |
| Bi | 0-15 | 0-15 | 0-15 | 0-15 |
| Cr | 0-15 | 0-15 | 0-15 | 1-10 |
| Ir | 1-34 | 0-25 | 3-27 | 0-15 |
| Nb | 0-15 | 3-27 | 0-15 | 0-15 |
| Ta | 0-15 | 0-15 | 1-34 | 0-15 |
| Ti | 0-15 | 0-15 | 0-15 | 0-15 |
| Y | 3-27 | 3-27 | 0-15 | 0-15 |
| Zr | 0-15 | 0-15 | 3-27 | 1-12 |
| Component/Wt. % | Ex. 87 | Ex. 88 | Ex. 89 | Ex. 90 |
| Re | 50-75% | 55-75% | 60-75% | 65-75% |
| Cr | 25-50% | 25-45% | 25-40% | 25-35% |
| Mo | 0-25% | 0-25% | 0-25% | 0-25% |
| Bi | 0-25% | 0-25% | 0-25% | 0-25% |
| Ir | 0-25% | 0-25% | 0-25% | 0-25% |
| Nb | 0-25% | 0-25% | 0-25% | 0-25% |
| Ta | 0-25% | 0-25% | 0-25% | 0-25% |
| V | 0-25% | 0-25% | 0-25% | 0-25% |
| W | 0-25% | 0-25% | 0-25% | 0-25% |
| Mn | 0-25% | 0-25% | 0-25% | 0-25% |
| Tc | 0-25% | 0-25% | 0-25% | 0-25% |
| Ru | 0-25% | 0-25% | 0-25% | 0-25% |
| Rh | 0-25% | 0-25% | 0-25% | 0-25% |
| Hf | 0-25% | 0-25% | 0-25% | 0-25% |
| Os | 0-25% | 0-25% | 0-25% | 0-25% |
| Cu | 0-25% | 0-25% | 0-25% | 0-25% |
| Ir | 0-25% | 0-25% | 0-25% | 0-25% |
| Ti | 0-25% | 0-25% | 0-25% | 0-25% |
| Y | 0-25% | 0-25% | 0-25% | 0-25% |
| Zr | 0-25% | 0-25% | 0-25% | 0-25% |
| Ag | 0-25% | 0-25% | 0-25% | 0-25% |
| Al | 0-25% | 0-25% | 0-25% | 0-22% |
| Co | 0-25% | 0-25% | 0-25% | 0-25% |
| Fe | 0-25% | 0-25% | 0-25% | 0-25% |
| Mg | 0-25% | 0-25% | 0-25% | 0-25% |
| Ni | 0-25% | 0-25% | 0-25% | 0-25% |
| Pt | 0-25% | 0-25% | 0-25% | 0-25% |
| Si | 0-25% | 0-25% | 0-25% | 0-25% |
| Sn | 0-25% | 0-25% | 0-25% | 0-25% |
| Component/Wt. % | Ex. 91 | Ex. 92 | Ex. 93 | Ex. 94 |
| Re | 50-72% | 55-72% | 60-72% | 65-72% |
| Cr | 28-50% | 28-45% | 28-40% | 28-35% |
| Mo | 0-25% | 0-25% | 0-25% | 0-25% |
| Bi | 0-10% | 0-10% | 0-10% | 0-10% |
| Ir | 0-10% | 0-10% | 0-10% | 0-10% |
| Nb | 0-10% | 0-10% | 0-10% | 0-10% |
| Ta | 0-10% | 0-10% | 0-10% | 0-10% |
| V | 0-10% | 0-10% | 0-10% | 0-10% |
| W | 0-10% | 0-10% | 0-10% | 0-10% |
| Mn | 0-10% | 0-10% | 0-10% | 0-10% |
| Tc | 0-10% | 0-10% | 0-10% | 0-10% |
| Ru | 0-10% | 0-10% | 0-10% | 0-10% |
| Rh | 0-10% | 0-10% | 0-10% | 0-10% |
| Hf | 0-10% | 0-10% | 0-10% | 0-10% |
| Os | 0-10% | 0-10% | 0-10% | 0-10% |
| Cu | 0-10% | 0-10% | 0-10% | 0-10% |
| Ir | 0-10% | 0-10% | 0-10% | 0-10% |
| Ti | 0-10% | 0-10% | 0-10% | 0-10% |
| Y | 0-10% | 0-10% | 0-10% | 0-10% |
| Zr | 0-10% | 0-10% | 0-10% | 0-10% |
| Ag | 0-10% | 0-10% | 0-10% | 0-10% |
| Al | 0-10% | 0-10% | 0-10% | 0-10% |
| Co | 0-10% | 0-10% | 0-10% | 0-10% |
| Fe | 0-10% | 0-10% | 0-10% | 0-10% |
| Mg | 0-10% | 0-10% | 0-10% | 0-10% |
| Ni | 0-10% | 0-10% | 0-10% | 0-10% |
| Pt | 0-10% | 0-10% | 0-10% | 0-10% |
| Si | 0-10% | 0-10% | 0-10% | 0-10% |
| Sn | 0-10% | 0-10% | 0-10% | 0-10% |
| Component/Wt. % | Ex. 95 | Ex. 96 | Ex. 97 | Ex. 98 |
| Re | 50-70% | 55-70% | 60-70% | 65-70% |
| Cr | 30-50% | 30-45% | 30-40% | 30-35% |
| Mo | 0-10% | 0-10% | 0-10% | 0-10% |
| Bi | 0-10% | 0-10% | 0-10% | 0-10% |
| Ir | 0-10% | 0-10% | 0-10% | 0-10% |
| Nb | 0-10% | 0-10% | 0-10% | 0-10% |
| Ta | 0-10% | 0-10% | 0-10% | 0-10% |
| V | 0-10% | 0-10% | 0-10% | 0-10% |
| W | 0-10% | 0-10% | 0-10% | 0-10% |
| Mn | 0-10% | 0-10% | 0-10% | 0-10% |
| Tc | 0-10% | 0-10% | 0-10% | 0-10% |
| Ru | 0-10% | 0-10% | 0-10% | 0-10% |
| Rh | 0-10% | 0-10% | 0-10% | 0-10% |
| Hf | 0-10% | 0-10% | 0-10% | 0-10% |
| Os | 0-10% | 0-10% | 0-10% | 0-10% |
| Cu | 0-10% | 0-10% | 0-10% | 0-10% |
| Ir | 0-10% | 0-10% | 0-10% | 0-10% |
| Ti | 0-10% | 0-10% | 0-10% | 0-10% |
| Y | 0-10% | 0-10% | 0-10% | 0-10% |
| Zr | 0-10% | 0-10% | 0-10% | 0-10% |
| Ag | 0-10% | 0-10% | 0-10% | 0-10% |
| Al | 0-10% | 0-10% | 0-10% | 0-10% |
| Co | 0-10% | 0-10% | 0-10% | 0-10% |
| Fe | 0-10% | 0-10% | 0-10% | 0-10% |
| Mg | 0-10% | 0-10% | 0-10% | 0-10% |
| Ni | 0-10% | 0-10% | 0-10% | 0-10% |
| Pt | 0-10% | 0-10% | 0-10% | 0-10% |
| Si | 0-10% | 0-10% | 0-10% | 0-10% |
| Sn | 0-10% | 0-10% | 0-10% | 0-10% |
| Component/Wt. % | Ex. 99 | Ex. 100 | Ex. 101 | Ex. 102 |
| Re | 50-67.5% | 55-67.5% | 60-67.5% | 65-67.5% |
| Cr | 32.5-50% | 32.5-45% | 32.5-40% | 32.5-35% |
| Mo | 0-10% | 0-10% | 0-10% | 0-10% |
| Bi | 0-10% | 0-10% | 0-10% | 0-10% |
| Ir | 0-10% | 0-10% | 0-10% | 0-10% |
| Nb | 0-10% | 0-10% | 0-10% | 0-10% |
| Ta | 0-10% | 0-10% | 0-10% | 0-10% |
| V | 0-10% | 0-10% | 0-10% | 0-10% |
| W | 0-10% | 0-10% | 0-10% | 0-10% |
| Mn | 0-10% | 0-10% | 0-10% | 0-10% |
| Tc | 0-10% | 0-10% | 0-10% | 0-10% |
| Ru | 0-10% | 0-10% | 0-10% | 0-10% |
| Rh | 0-10% | 0-10% | 0-10% | 0-10% |
| Hf | 0-10% | 0-10% | 0-10% | 0-10% |
| Os | 0-10% | 0-10% | 0-10% | 0-10% |
| Cu | 0-10% | 0-10% | 0-10% | 0-10% |
| Ir | 0-10% | 0-10% | 0-10% | 0-10% |
| Ti | 0-10% | 0-10% | 0-10% | 0-10% |
| Y | 0-10% | 0-10% | 0-10% | 0-10% |
| Zr | 0-10% | 0-10% | 0-10% | 0-10% |
| Ag | 0-10% | 0-10% | 0-10% | 0-10% |
| Al | 0-10% | 0-10% | 0-10% | 0-10% |
| Co | 0-10% | 0-10% | 0-10% | 0-10% |
| Fe | 0-10% | 0-10% | 0-10% | 0-10% |
| Mg | 0-10% | 0-10% | 0-10% | 0-10% |
| Ni | 0-10% | 0-10% | 0-10% | 0-10% |
| Pt | 0-10% | 0-10% | 0-10% | 0-10% |
| Si | 0-10% | 0-10% | 0-10% | 0-10% |
| Sn | 0-10% | 0-10% | 0-10% | 0-10% |
| Component/Wt. % | Ex. 103 | Ex. 104 | Ex. 105 | Ex. 106 |
| Re | 50-67.5% | 55-67.5% | 60-67.5% | 65-67.5% |
| Cr | 32.5-50% | 32.5-45% | 32.5-40% | 32.5-35% |
| Mo | 0-5% | 0-5% | 0-5% | 0-5% |
| Bi | 0-5% | 0-5% | 0-5% | 0-5% |
| Ir | 0-5% | 0-5% | 0-5% | 0-5% |
| Nb | 0-5% | 0-5% | 0-5% | 0-5% |
| Ta | 0-5% | 0-5% | 0-5% | 0-5% |
| V | 0-5% | 0-5% | 0-5% | 0-5% |
| W | 0-5% | 0-5% | 0-5% | 0-5% |
| Mn | 0-5% | 0-5% | 0-5% | 0-5% |
| Tc | 0-5% | 0-5% | 0-5% | 0-5% |
| Ru | 0-5% | 0-5% | 0-5% | 0-5% |
| Rh | 0-5% | 0-5% | 0-5% | 0-5% |
| Hf | 0-5% | 0-5% | 0-5% | 0-5% |
| Os | 0-5% | 0-5% | 0-5% | 0-5% |
| Cu | 0-5% | 0-5% | 0-5% | 0-5% |
| Ir | 0-5% | 0-5% | 0-5% | 0-5% |
| Ti | 0-5% | 0-5% | 0-5% | 0-5% |
| Y | 0-5% | 0-5% | 0-5% | 0-5% |
| Zr | 0-5% | 0-5% | 0-5% | 0-5% |
| Ag | 0-5% | 0-5% | 0-5% | 0-5% |
| Al | 0-5% | 0-5% | 0-5% | 0-5% |
| Co | 0-5% | 0-5% | 0-5% | 0-5% |
| Fe | 0-5% | 0-5% | 0-5% | 0-5% |
| Mg | 0-5% | 0-5% | 0-5% | 0-5% |
| Ni | 0-5% | 0-5% | 0-5% | 0-5% |
| Pt | 0-5% | 0-5% | 0-5% | 0-5% |
| Si | 0-5% | 0-5% | 0-5% | 0-5% |
| Sn | 0-5% | 0-5% | 0-5% | 0-5% |
| Component/Wt. % | Ex. 107 | Ex. 108 | Ex. 109 | Ex. 110 |
| Re | 50-75% | 55-72% | 60-70% | 62-70% |
| Cr | 24-49% | 27-44% | 29-39% | 29-37% |
| Mo | 1-15% | 1-10% | 1-8% | 1-5% |
| Bi | 0-15% | 0-10% | 0-8% | 0-5% |
| Cr | 0-15% | 0-10% | 0-8% | 0-5% |
| Nb | 0-15% | 0-10% | 0-8% | 0-5% |
| Ta | 0-15% | 0-10% | 0-8% | 0-5% |
| V | 0-15% | 0-10% | 0-8% | 0-5% |
| W | 0-15% | 0-10% | 0-8% | 0-5% |
| Mn | 0-15% | 0-10% | 0-8% | 0-5% |
| Tc | 0-15% | 0-10% | 0-8% | 0-5% |
| Ru | 0-15% | 0-10% | 0-8% | 0-5% |
| Rh | 0-15% | 0-10% | 0-8% | 0-5% |
| Hf | 0-15% | 0-10% | 0-8% | 0-5% |
| Os | 0-15% | 0-10% | 0-8% | 0-5% |
| Cu | 0-15% | 0-10% | 0-8% | 0-5% |
| Ir | 0-15% | 0-10% | 0-8% | 0-5% |
| Ti | 0-15% | 0-10% | 0-8% | 0-5% |
| Y | 0-15% | 0-10% | 0-8% | 0-5% |
| Zr | 0-15% | 0-10% | 0-8% | 0-5% |
| Ag | 0-15% | 0-10% | 0-8% | 0-5% |
| Al | 0-15% | 0-10% | 0-8% | 0-5% |
| Co | 0-15% | 0-10% | 0-8% | 0-5% |
| Fe | 0-15% | 0-10% | 0-8% | 0-5% |
| Mg | 0-15% | 0-10% | 0-8% | 0-5% |
| Ni | 0-15% | 0-10% | 0-8% | 0-5% |
| Pt | 0-15% | 0-10% | 0-8% | 0-5% |
| Si | 0-15% | 0-10% | 0-8% | 0-5% |
| Sn | 0-15% | 0-10% | 0-8% | 0-5% |
| Component/Wt. % | Ex. 111 | Ex. 112 | Ex. 113 | Ex. 114 |
| Mo | 40-95% | 40-95% | 40-95% | 40-95% |
| Co | ≤0.002% | ≤0.002% | ≤0.002% | ≤0.002% |
| Fe | ≤0.02% | ≤0.02% | ≤0.02% | ≤0.02% |
| Hf | 0.1-2.5% | 0-2.5% | 0-2.5% | 0-2.5% |
| Os | ≤1% | ≤1% | ≤1% | ≤1% |
| Nb | ≤0.01% | ≤0.01% | ≤0.01% | ≤0.01% |
| Pt | ≤1% | ≤1% | ≤1% | ≤1% |
| Re | 5-40% | 5-40% | 5-40% | 5-40% |
| Sn | ≤0.002% | ≤0.002% | ≤0.002% | ≤0.002% |
| Ta | 0-50% | 0-50% | 0-50% | 0-50% |
| Tc | ≤1% | ≤1% | ≤1% | ≤1% |
| Ti | ≤1% | ≤1% | ≤1% | ≤1% |
| V | ≤1% | ≤1% | ≤1% | ≤1% |
| W | 0-50% | 0-50% | 0-50% | 0.5-50% |
| Zr | ≤1% | ≤1% | ≤1% | ≤1% |
| Ag | 0-5% | 0-5% | 0-5% | 0-5% |
| Al | 0-5% | 0-5% | 0-5% | 0-5% |
| Co | 0-5% | 0-5% | 0-5% | 0-5% |
| Mg | 0-5% | 0-5% | 0-5% | 0-5% |
| Ni | 0-5% | 0-5% | 0-5% | 0-5% |
| Si | 0-5% | 0-5% | 0-5% | 0-5% |
| Sn | 0-5% | 0-5% | 0-5% | 0-5% |
| Component/Wt. % | Ex. 115 | Ex. 116 | Ex. 117 | |
| Mo | 40-95% | 40-95% | 40-95% | |
| Co | ≤0.002% | ≤0.002% | ≤0.002% | |
| Hf | 0-2.5% | 0-2.5% | 0-2.5% | |
| Os | ≤1% | ≤1% | ≤1% | |
| Nb | ≤0.01% | ≤0.01% | ≤0.01% | |
| Pt | ≤1% | ≤1% | ≤1% | |
| Re | 5-40% | 5-40% | 5-40% | |
| Sn | ≤0.002% | ≤0.002% | ≤0.002% | |
| Ta | 0-50% | 0.5-50% | 0-50% | |
| Tc | ≤1% | ≤1% | ≤1% | |
| Ti | ≤1% | ≤1% | ≤1% | |
| V | ≤1% | ≤1% | ≤1% | |
| W | 0-50% | 0-50% | 0-50% | |
| Ag | 0-5% | 0-5% | 0-5% | |
| Al | 0-5% | 0-5% | 0-5% | |
| Fe | 0-5% | 0-5% | 0-5% | |
| Mg | 0-5% | 0-5% | 0-5% | |
| Ni | 0-5% | 0-5% | 0-5% | |
| Si | 0-5% | 0-5% | 0-5% | |
| Component/Wt. % | Ex. 118 | Ex. 119 | Ex. 120 | |
| Mo | 60-95% | 60-95% | 60-90% | |
| Co | ≤0.002% | ≤0.002% | ≤0.002% | |
| Hf | 0-2.5% | 0-2.5% | 0-2.5% | |
| O | ≤0.06% | ≤0.06% | ≤0.06% | |
| Os | ≤1% | ≤1% | ≤1% | |
| Nb | ≤0.01% | ≤0.01% | <0.01% | |
| Pt | ≤1% | ≤1% | ≤1% | |
| Re | 5-40% | 5-40% | 10-40% | |
| Sn | ≤0.002% | ≤0.002% | ≤0.002% | |
| Ta | 0-50% | 0.5-50% | 0-50% | |
| Tc | ≤1% | ≤1% | ≤1% | |
| Ti | ≤1% | ≤1% | ≤1% | |
| V | ≤1% | ≤1% | ≤1% | |
| W | 0-50% | 0-50% | 0-50% | |
| Ag | 0-5% | 0-5% | 0-5% | |
| Al | 0-5% | 0-5% | 0-5% | |
| Fe | 0-5% | 0-5% | 0-5% | |
| Mg | 0-5% | 0-5% | 0-5% | |
| Ni | 0-5% | 0-5% | 0-5% | |
| Si | 0-5% | 0-5% | 0-5% | |
| Component/Wt. % | Ex. 121 | Ex. 122 | Ex. 123 | Ex.124 |
| Mo | 60-95% | 60-95% | 50-95% | 40-80% |
| Hf | 0.8-1.4% | 0-2% | 0-2.5% | 0-2.5% |
| Re | 5-40% | 5-40% | 5-40% | 5-40% |
| Ta | 0-2% | 0-2% | 0-50% | 0-50% |
| W | 0-2% | 0-2% | 0-50% | 20-50% |
| Component/Wt. % | Ex. 125 | Ex. 126 | Ex. 127 | |
| Mo | 97-95% | 50-90% | 60-95% | |
| Hf | 0-2.5% | 0-2.5% | 0-2.5% | |
| Re | 5-30 | 5-40% | 5-40% | |
| Ta | 0-3% | 10-50% | 0-40% | |
| W | 0-3% | 0-50% | 0-40% | |
| Component/Wt. % | Ex. 128 | Ex. 129 | Ex. 130 | |
| W | 20-95% | 60-95% | 20-80% | |
| Re | 5-47.5% | 5-40% | 5-47.5% | |
| Mo | 0-47.5% | <0.5% | 1-47.5% | |
| Cu | <0.5% | <0.5% | <0.5% | |
| Co | ≤0.002% | ≤0.002% | ≤0.002% | |
| Fe | ≤0.02% | ≤0.02% | ≤0.02% | |
| Hf | <0.5% | <0.5% | <0.5% | |
| Os | <0.5% | <0.5% | <0.5% | |
| Nb | ≤0.01% | ≤0.01% | ≤0.01% | |
| Pt | <0.5% | <0.5% | <0.5% | |
| Sn | ≤0.002% | ≤0.002% | ≤0.002% | |
| Ta | <0.5% | <0.5% | <0.5% | |
| Tc | <0.5% | <0.5% | <0.5% | |
| Ti | <0.5% | <0.5% | <0.5% | |
| V | <0.5% | <0.5% | <0.5% | |
| Zr | <0.5% | <0.5% | <0.5% | |
| Ag | 0-5% | 0-5% | 0-5% | |
| Al | 0-5% | 0-5% | 0-5% | |
| Fe | 0-5% | 0-5% | 0-5% | |
| Mg | 0-5% | 0-5% | 0-5% | |
| Ni | 0-5% | 0-5% | 0-5% | |
| Si | 0-5% | 0-5% | 0-5% | |
| Component/Wt. % | Ex. 131 | Ex. 132 | Ex. 133 | Ex. 134 |
| W | 1-94.9% | 1-94.9% | 1-94.9% | 10-95% |
| Cu | 0.1-94% | 0.1-94% | 0.1-94% | 1-84% |
| Co | ≤0.002% | ≤0.002% | ≤0.002% | ≤0.002% |
| Fe | ≤0.02% | ≤0.02% | ≤0.02% | ≤0.02% |
| Hf | 0.1-2.5% | 0-2.5% | 0-2.5% | 0-2.5% |
| Os | ≤1% | ≤1% | ≤1% | ≤1% |
| Mo | 0-5% | 0.1-3% | 0-2% | 0-3% |
| Nb | ≤0.01% | <0.01% | ≤0.01% | ≤0.01% |
| Pt | ≤1% | ≤1% | ≤1% | ≤1% |
| Re | 5-40% | 5-40% | 5-40% | 6-40% |
| Sn | ≤0.002% | ≤0.002% | ≤0.002% | ≤0.002% |
| Ta | 0-50% | 0-50% | 0-50% | 0-50% |
| Tc | ≤1% | ≤1% | ≤1% | ≤1% |
| Ti | ≤1% | ≤1% | ≤1% | ≤1% |
| V | ≤1% | ≤1% | ≤1% | ≤1% |
| Zr | ≤1% | ≤1% | ≤1% | ≤1% |
| Ag | 0-5% | 0-5% | 0-5% | 0-5% |
| Al | 0-5% | 0-5% | 0-5% | 0-5% |
| Fe | 0-5% | 0-5% | 0-5% | 0-5% |
| Mg | 0-5% | 0-5% | 0-5% | 0-5% |
| Ni | 0-5% | 0-5% | 0-5% | 0-5% |
| Si | 0-5% | 0-5% | 0-5% | 0-5% |
| Component/Wt. % | Ex. 135 | Ex. 136 | Ex. 137 | |
| W | 20-96% | 25-92% | 30-88% | |
| Cu | 2-74% | 2-68% | 5-62% | |
| Co | ≤0.002% | ≤0.002% | ≤0.002% | |
| Hf | 0-2.5% | 0-2.5% | 0-2.5% | |
| Os | ≤1% | ≤1% | ≤1% | |
| Mo | 0-3% | 0-2% | 0-1% | |
| Nb | ≤0.01% | ≤0.01% | ≤0.01% | |
| Pt | ≤1% | ≤1% | ≤1% | |
| Re | 6-40% | 7-40% | 8-40% | |
| Sn | ≤0.002% | ≤0.002% | ≤0.002% | |
| Ta | 0-50% | 0.5-50% | 0-50% | |
| Tc | ≤1% | ≤1% | ≤1% | |
| Ti | ≤1% | ≤1% | ≤1% | |
| V | ≤1% | ≤1% | ≤1% | |
| Ag | 0-5% | 0-5% | 0-5% | |
| Al | 0-5% | 0-5% | 0-5% | |
| Fe | 0-5% | 0-5% | 0-5% | |
| Mg | 0-5% | 0-5% | 0-5% | |
| Ni | 0-5% | 0-5% | 0-5% | |
| Si | 0-5% | 0-5% | 0-5% | |
| Component/Wt. % | Ex. 138 | Ex. 139 | Ex. 140 | Ex. 141 |
| W | 25-88% | 35-87% | 40-86% | 50-80% |
| Cu | 5-68% | 5-57% | 5-51% | 5-40% |
| Hf | 0.8-1.4% | 0-2.5% | 0-2.5% | 0-2.5% |
| Re | 0-40% | 0-40% | 0-40% | 0-40% |
| Ta | 0-50% | 0-50% | 0-50% | 0-50% |
| Component/Wt. % | Ex. 142 | Ex. 143 | Ex. 144 | |
| W | 55-88% | 60-87% | 70-86% | |
| Cu | 1-34% | 1-28% | 1-17% | |
| C | 0-0.15% | 0-0.15% | 0-0.15% | |
| Hf | 0-2.5% | 0-2.5% | 0-2.5% | |
| Re | 11-40% | 12-40% | 13-40% | |
| Ta | 0-50% | 10-50% | 0-50% | |
| W | 0-50% | 0-50% | 0-50% | |
| Component/Wt. % | Ex. 145 | Ex. 146 | Ex. 147 | |
| Ti | 55-66% | 65-76% | 70-76% | |
| Mo | 20-41% | 20-31% | 20-26% | |
| Re | 4-20% | 4-20% | 4-20% | |
| Yt | <0.5% | <0.5% | <0.5% | |
| Nb | <0.5% | <0.5% | <0.5% | |
| Co | <0.5% | <0.5% | <0.5% | |
| Cr | <0.5% | <0.5% | <0.5% | |
| Zr | <0.5% | <0.5% | <0.5% | |
| Component/Wt. % | Ex. 148 | Ex. 149 | Ex. 150 | |
| W | 20-95% | 60-85% | 20-84% | |
| Re | 5-47.5% | 15-40% | 5-47.5% | |
| Mo | 0-47.5% | <0.5% | 1-47.5% | |
| Component/Wt. % | Ex. 151 | Ex. 152 | Ex. 153 | |
| W | 50.1-93% | 65-92% | 70-90% | |
| Re | 7-40% | 8-35% | 9-30% | |
| Mo | 0-40% | <0.5% | 1-30% | |
| Component/Wt. % | Ex. 154 | Ex. 155 | Ex. 156 | |
| W | 20-49% | 20-49% | 20-49% | |
| Re | 5-40% | 5-40% | 5-39% | |
| Mo | 20-60% | 30-60% | 40-60% | |
| Component/Wt. % | Ex. 157 | Ex. 158 | Ex. 159 | |
| W | 20-40% | 20-35% | 20-30% | |
| Re | 7-40% | 10-40% | 25-40% | |
| Mo | 0-40% | 10-40% | 25-40% | |
| Component/Wt. % | Ex. 160 | Ex. 161 | Ex. 162 | |
| W | 20-95% | 60-93% | 20-80% | |
| Re | 5-47.5% | 7-40% | 5-47.5% | |
| Mo | 0-47.5% | <0.5% | 1-47.5% | |
| Cu | <0.5% | <0.5% | <0.5% | |
| Co | ≤0.002% | ≤0.002% | ≤0.002% | |
| Fe | ≤0.02% | ≤0.02% | ≤0.02% | |
| Hf | <0.5% | <0.5% | <0.5% | |
| Os | <0.5% | <0.5% | <0.5% | |
| Nb | ≤0.01% | ≤0.01% | ≤0.01% | |
| Pt | <0.5% | <0.5% | <0.5% | |
| Sn | ≤0.002% | ≤0.002% | ≤0.002% | |
| Ta | <0.5% | <0.5% | <0.5% | |
| Tc | <0.5% | <0.5% | <0.5% | |
| Ti | <0.5% | <0.5% | <0.5% | |
| V | <0.5% | <0.5% | <0.5% | |
| Zr | <0.5% | <0.5% | <0.5% | |
| Ag | 0-5% | 0-5% | 0-5% | |
| Al | 0-5% | 0-5% | 0-5% | |
| Fe | 0-5% | 0-5% | 0-5% | |
| Mg | 0-5% | 0-5% | 0-5% | |
| Ni | 0-5% | 0-5% | 0-5% | |
| Si | 0-5% | 0-5% | 0-5% | |
| Component/Wt. % | Ex. 163 | Ex. 164 | Ex. 165 | Ex. 166 |
| Ag | 0-10% | 0-10% | 0-10% | 0-10% |
| Al | 0-10% | 0-10% | 0-10% | 2-10% |
| B | 0-10% | 0-10% | 0-10% | 0-10% |
| Bi | 0-10% | 0-10% | 0-10% | 0-10% |
| Cr | 2-30% | 10-30% | 0-20% | 0-20% |
| Cu | 0-10% | 0-10% | 0-10% | 0-10% |
| Co | 0-10% | 32-70% | 0-10% | 0-10% |
| Fe | 50-80% | 0-20% | 0-10% | 0-10% |
| Hf | 0-10% | 0-10% | 0-10% | 0-10% |
| Ir | 0-10% | 0-10% | 0-10% | 0-10% |
| La | 0-10% | 0-10% | 0-10% | 0-10% |
| Mg | 0-10% | 0-10% | 0-10% | 0-10% |
| Mn | 0-20% | 0-10% | 0-10% | 0-10% |
| Mo | 0-10% | 0-30% | 0-16% | 0-16% |
| Nb | 0-10% | 0-10% | 0-10% | 0-10% |
| Ni | 0.1-30% | 0.1-40% | 0-10% | 0-10% |
| Os | 0-10% | 0-10% | 0-10% | 0-10% |
| Pt | 0-10% | 0-10% | 0-10% | 0-10% |
| Re | 5-40% | 4.8-40% | 4.5-80% | 4.5-80% |
| Rh | 0-10% | 0-10% | 0-10% | 0-10% |
| Se | 0-10% | 0-10% | 0-10% | 0-10% |
| Si | 0-10% | 0-10% | 0-10% | 0-10% |
| Sn | 0-10% | 0-10% | 0-12% | 0-12% |
| Ta | 0-10% | 0-10% | 0-10% | 0-10% |
| Tc | 0-10% | 0-10% | 0-10% | 0-10% |
| Ti | 0-10% | 0-10% | 70-91.5% | 70-91.5% |
| V | 0-10% | 0-10% | 0-10% | 0.01-10% |
| W | 0-10% | 0-20% | 0-10% | 0-10% |
| Y | 0-10% | 0-10% | 0-10% | 0-10% |
| Zr | 0-10% | 0-10% | 0-10% | 0-10% |
| Component/Wt. % | Ex. 167 | Ex. 168 | Ex. 169 | Ex. 170 |
| Ag | 0-10% | 0-10% | 0-10% | 0-10% |
| Al | 0-10% | 0-10% | 0-10% | 0-10% |
| B | 0-10% | 0-10% | 0-10% | 0-10% |
| Bi | 0-10% | 0-10% | 0-10% | 0-10% |
| Cr | 0-10% | 0-20% | 0-20% | 0-10% |
| Cu | 0-10% | 0-10% | 0-50% | 0-10% |
| Co | 0-10% | 0-10% | 0-10% | 0-10% |
| Fe | 0-10% | 0-10% | 0-10% | 0-10% |
| Hf | 0-10% | 0-10% | 0-10% | 0-10% |
| Ir | 0-10% | 0-10% | 0-10% | 0-12% |
| La | 0-10% | 0-10% | 0-10% | 0-10% |
| Mg | 0-10% | 0-10% | 0-10% | 0-10% |
| Mn | 0-10% | 0-10% | 0-10% | 0-10% |
| Mo | 0-55% | 40-93% | 0-50% | 0-20% |
| Nb | 0-10% | 0-10% | 0-10% | 40-85% |
| Ni | 0-45% | 0-10% | 0-10% | 0-10% |
| Os | 0-10% | 0-10% | 0-10% | 0-10% |
| Pt | 0-10% | 0-10% | 0-10% | 0-10% |
| Re | 14-40% | 7-40% | 7-40% | 7-40% |
| Rh | 0-10% | 0-10% | 0-10% | 0-10% |
| Se | 0-10% | 0-10% | 0-10% | 0-10% |
| Si | 0-10% | 0-10% | 0-10% | 0-10% |
| Sn | 0-10% | 0-10% | 0-10% | 0-10% |
| Ta | 35-84% | 0-50% | 0-50% | 0-35% |
| Tc | 0-10% | 0-10% | 0-10% | 0-10% |
| Ti | 0-10% | 0-10% | 0-10% | 0-10% |
| V | 0-10% | 0-10% | 0-10% | 0-10% |
| W | 0.1-25% | 0-50% | 14-10% | 0-15% |
| Y | 0-10% | 0-10% | 0-10% | 0-10% |
| Zr | 0-10% | 0-10% | 0-50% | 0-10% |
| Component/Wt. % | Ex. 171 | Ex. 172 | Ex. 173 | Ex. 174 |
| Ag | 0-10% | 0-10% | 0-5% | 0-5% |
| Al | 0-10% | 0-10% | 0-5% | 5-7% |
| B | 0-10% | 0-10% | 0-5% | 0-5% |
| Bi | 0-10% | 0-10% | 0-5% | 0-5% |
| Cr | 0-10% | 1-95% | 12-28% | 0-5% |
| Cu | 0-10% | 0-10% | 0-5% | 0-5% |
| Co | 0-10% | 0-10% | 36-68% | 0-5% |
| Fe | 0-10% | 0-10% | 0-18% | 0-5% |
| Hf | 0-10% | 0-10% | 0-5% | 0-5% |
| Ir | 0-10% | 0-10% | 0-5% | 0-5% |
| La | 0-10% | 0-10% | 0-5% | 0-5% |
| Mg | 0-10% | 0-10% | 0-5% | 0-5% |
| Mn | 0-10% | 0-10% | 0-5% | 0-5% |
| Mo | 0-10% | 0-20% | 0-12% | 0-5% |
| Nb | 0-10% | 0-10% | 0-5% | 0-5% |
| Ni | 30-58% | 0-10% | 9-36% | 0-5% |
| Os | 0-10% | 0-10% | 0-5% | 0-5% |
| Pt | 0-10% | 0-10% | 0-5% | 0-5% |
| Re | 5-40% | 5-40% | 4.8-40% | 4.5-40% |
| Rh | 0-10% | 0-10% | 0-5% | 0-5% |
| Se | 0-10% | 0-10% | 0-5% | 0-5% |
| Si | 0-10% | 0-10% | 0-5% | 0-5% |
| Sn | 0-10% | 0-10% | 0-5% | 0-5% |
| Ta | 0-10% | 0-10% | 0-5% | 0-5% |
| Tc | 0-10% | 0-10% | 0-5% | 0-5% |
| Ti | 30-58% | 0-40% | 0-5% | 70-91.5% |
| V | 0-10% | 0-10% | 0-5% | 3-6% |
| W | 0-10% | 0-10% | 0-16% | 0-5% |
| Y | 0-10% | 0-10% | 0-5% | 0-5% |
| Zr | 0-10% | 0-20% | 0-5% | 0-5% |
| Component/Wt. % | Ex. 175 | Ex. 176 | Ex. 177 | Ex. 178 |
| Ag | 0-8% | 0-8% | 0-8% | 0-8% |
| Al | 0-8% | 0-8% | 0-8% | 2-10% |
| B | 0-8% | 0-8% | 0-8% | 0-8% |
| Bi | 0-8% | 0-8% | 0-8% | 0-8% |
| Cr | 2-30% | 10-30% | 0-20% | 0-20% |
| Cu | 0-8% | 0-8% | 0-8% | 0-8% |
| Co | 0-8% | 32-70% | 0-8% | 0-8% |
| Fe | 50-80% | 0-20% | 0-8% | 0-8% |
| Hf | 0-8% | 0-8% | 0-8% | 0-8% |
| Ir | 0-8% | 0-8% | 0-8% | 0-8% |
| La | 0-8% | 0-8% | 0-8% | 0-8% |
| Mg | 0-8% | 0-8% | 0-8% | 0-8% |
| Mn | 0-20% | 0-8% | 0-8% | 0-8% |
| Mo | 0-8% | 0-30% | 0-16% | 0-16% |
| Nb | 0-8% | 0-8% | 0-8% | 0-8% |
| Ni | 0.1-30% | 0.1-40% | 0-8% | 0-8% |
| Os | 0-8% | 0-8% | 0-8% | 0-8% |
| Pt | 0-8% | 0-8% | 0-8% | 0-8% |
| Re | 5-40% | 4.8-40% | 4.5-80% | 4.5-80% |
| Rh | 0-8% | 0-8% | 0-8% | 0-8% |
| Se | 0-8% | 0-8% | 0-8% | 0-8% |
| Si | 0-8% | 0-8% | 0-8% | 0-8% |
| Sn | 0-8% | 0-8% | 0-12% | 0-12% |
| Ta | 0-8% | 0-8% | 0-8% | 0-8% |
| Tc | 0-8% | 0-8% | 0-8% | 0-8% |
| Ti | 0-8% | 0-8% | 70-91.5% | 70-91.5% |
| V | 0-8% | 0-8% | 0-8% | 0.01-10% |
| W | 0-8% | 0-20% | 0-8% | 0-8% |
| Y | 0-8% | 0-8% | 0-8% | 0-8% |
| Zr | 0-8% | 0-8% | 0-8% | 0-8% |
| Component/Wt. % | Ex. 179 | Ex. 180 | Ex. 181 | Ex. 182 |
| Ag | 0-8% | 0-8% | 0-8% | 0-8% |
| Al | 0-8% | 0-8% | 0-8% | 0-8% |
| B | 0-8% | 0-8% | 0-8% | 0-8% |
| Bi | 0-8% | 0-8% | 0-8% | 0-8% |
| Cr | 0-8% | 0-20% | 0-20% | 0-8% |
| Cu | 0-8% | 0-8% | 0-50% | 0-8% |
| Co | 0-8% | 0-8% | 0-8% | 0-8% |
| Fe | 0-8% | 0-8% | 0-8% | 0-8% |
| Hf | 0-8% | 0-8% | 0-8% | 0-8% |
| Ir | 0-8% | 0-8% | 0-8% | 0-12% |
| La | 0-8% | 0-8% | 0-8% | 0-8% |
| Mg | 0-8% | 0-8% | 0-8% | 0-8% |
| Mn | 0-8% | 0-8% | 0-8% | 0-8% |
| Mo | 0-55% | 40-93% | 0-50% | 0-20% |
| Nb | 0-8% | 0-8% | 0-8% | 40-85% |
| Ni | 0-45% | 0-8% | 0-8% | 0-8% |
| Os | 0-8% | 0-8% | 0-8% | 0-8% |
| Pt | 0-8% | 0-8% | 0-8% | 0-8% |
| Re | 14-40% | 7-40% | 7-40% | 7-40% |
| Rh | 0-8% | 0-8% | 0-8% | 0-8% |
| Se | 0-8% | 0-8% | 0-8% | 0-8% |
| Si | 0-8% | 0-8% | 0-8% | 0-8% |
| Sn | 0-8% | 0-8% | 0-8% | 0-8% |
| Ta | 35-84% | 0-50% | 0-50% | 0-35% |
| Tc | 0-8% | 0-8% | 0-8% | 0-8% |
| Ti | 0-8% | 0-8% | 0-8% | 0-8% |
| V | 0-8% | 0-8% | 0-8% | 0-8% |
| W | 0.1-25% | 0-50% | 14-10% | 0-15% |
| Y | 0-8% | 0-8% | 0-8% | 0-8% |
| Zr | 0-8% | 0-8% | 0-50% | 0-8% |
| Component/Wt. % | Ex. 183 | Ex. 184 | Ex. 185 | Ex. 186 |
| Ag | 0-5% | 0-5% | 0-5% | 0-5% |
| Al | 0-5% | 0-5% | 0-5% | 5-7% |
| B | 0-5% | 0-5% | 0-5% | 0-5% |
| Bi | 0-5% | 0-5% | 0-5% | 0-5% |
| Cr | 0-5% | 1-95% | 12-28% | 0-5% |
| Cu | 0-5% | 0-5% | 0-5% | 0-5% |
| Co | 0-5% | 0-5% | 36-68% | 0-5% |
| Fe | 0-5% | 0-5% | 0-18% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-5% | 0-20% | 0-12% | 0-5% |
| Nb | 0-5% | 0-5% | 0-5% | 0-5% |
| Ni | 30-58% | 0-5% | 9-36% | 0-5% |
| Os | 0-5% | 0-5% | 0-5% | 0-5% |
| Pt | 0-5% | 0-5% | 0-5% | 0-5% |
| Re | 5-40% | 5-40% | 4.8-40% | 4.5-40% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 0-5% | 0-5% |
| Tc | 0-5% | 0-5% | 0-5% | 0-5% |
| Ti | 30-58% | 0-40% | 0-5% | 70-91.5% |
| V | 0-5% | 0-5% | 0-5% | 3-6% |
| W | 0-5% | 0-5% | 0-16% | 0-5% |
| Y | 0-5% | 0-5% | 0-5% | 0-5% |
| Zr | 0-5% | 0-20% | 0-5% | 0-5% |
| Component/Wt. % | Ex. 187 | Ex. 188 | Ex. 189 | Ex. 190 |
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 0-5% | 0-5% | 0-5% |
| Cu | 0-5% | 0-5% | 0-5% | 0-5% |
| Co | 0-5% | 0-5% | 0-5% | 0-5% |
| Fe | 0-5% | 0-5% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 1-15% | 2-10% | 3-8% | 0-5% |
| Nb | 0-5% | 0-5% | 0-5% | 20-45% |
| Ni | 0-5% | 0-5% | 0-5% | 0-5% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 0-5% | 1-15% |
| Tc | 0-5% | 0-5% | 0-5% | 0-5% |
| Ti | 51-70% | 51-70% | 55-70% | 51-70% |
| V | 0-5% | 0-5% | 0-5% | 0-5% |
| W | 0-5% | 0-5% | 0-5% | 0-5% |
| Y | 0-5% | 0-5% | 0-5% | 0-5% |
| Zr | 20-40% | 22-38% | 27-33% | 1-15% |
| Component/Wt. % | Ex. 191 | Ex. 192 | Ex. 193 | Ex. 194 |
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 0-5% | 0-5% | 0-5% |
| Cu | 0-5% | 0-5% | 0-5% | 0-5% |
| Co | 0-5% | 0-5% | 0-5% | 0-5% |
| Fe | 0-5% | 0-5% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-5% | 0-5% | 0-5% | 0-5% |
| Nb | 25-40% | 30-40% | 25-40% | 26-32% |
| Ni | 0-5% | 0-5% | 0-5% | 0-5% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 2-8% | 3-6% | 5-15% | 10-14% |
| Tc | 0-5% | 0-5% | 0-5% | 0-5% |
| Ti | 51-70% | 52-63% | 51-68% | 51-62% |
| V | 0-5% | 0-5% | 0-5% | 0-5% |
| W | 0-5% | 0-5% | 0-5% | 0-5% |
| Y | 0-5% | 0-5% | 0-5% | 0-5% |
| Zr | 2-12% | 4-8% | 2-8% | 2-6% |
| Component/Wt. % | Ex. 195 | Ex. 196 | Ex. 197 | Ex. 198 |
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 5-35% | 10-30% | 15-25% |
| Cu | 0-5% | 0-5% | 0-5% | 0-5% |
| Co | 0-5% | 20-55% | 25-50% | 35-45% |
| Fe | 0-5% | 3-25% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-5% | 2-15% | 3-12% | 4-9% |
| Nb | 30-40% | 0-5% | 0-5% | 0-5% |
| Ni | 0-5% | 4-23% | 5-20% | 10-18% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 1-3% | 0-5% | 0-5% | 0-5% |
| Tc | 0-5% | 0-5% | 0-5% | 0-5% |
| Ti | 51-67% | 0-5% | 0-5% | 0-5% |
| V | 0-5% | 0-5% | 0-5% | 0-5% |
| W | 0-5% | 0-5% | 0-5% | 0-5% |
| Y | 0-5% | 0-5% | 0-5% | 0-5% |
| Zr | 2-5% | 0-5% | 0-5% | 0-5% |
| Component/Wt. % | Ex. 199 | Ex. 200 | Ex. 201 | Ex. 202 |
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 0-5% | 0-5% | 0-5% |
| Cu | 0-5% | 0-5% | 0-5% | 0-5% |
| Co | 0-5% | 0-5% | 0-5% | 0-5% |
| Fe | 0-5% | 0-5% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 30-65% | 40-60% | 45-55% | 0-5% |
| Nb | 0-5% | 0-5% | 0-5% | 55-99.75% |
| Ni | 0-5% | 0-5% | 0-5% | 0-5% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 0-5% | 0-5% |
| Tc | 0-5% | 0-5% | 0-5% | 0-5% |
| Ti | 0-5% | 0-5% | 0-5% | 0-5% |
| V | 0-5% | 0-5% | 0-5% | 0-5% |
| W | 0-5% | 0-5% | 0-5% | 0-5% |
| Y | 0-5% | 0-5% | 0-5% | 0-5% |
| Zr | 30-56% | 40-60% | 45-55% | 0.25-45% |
| Component/Wt. % | Ex. 203 | Ex. 204 | Ex. 205 | Ex. 206 |
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 0-5% | 0-5% | 0-5% |
| Cu | 0-5% | 0-5% | 0-5% | 0-5% |
| Co | 0-5% | 0-5% | 0-5% | 0-5% |
| Fe | 0-5% | 0-5% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-5% | 0-5% | 0-5% | 0-5% |
| Nb | 75-99.5% | 95-99.25% | 55-78.5% | 68-74.25% |
| Ni | 0-5% | 0-5% | 0-5% | 0-5% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 20-35% | 25-30% |
| Tc | 0-5% | 0-5% | 0-5% | 0-5% |
| Ti | 0-5% | 0-5% | 0-5% | 0-5% |
| V | 0-5% | 0-5% | 0-5% | 0-5% |
| W | 0-5% | 0-5% | 1-8% | 0-5% |
| Y | 0-5% | 0-5% | 0-5% | 0-5% |
| Zr | 0.5-25% | 0.75-5% | 0.5-5% | 0.75-3% |
| Element/Wt. % | Ex. 207 | Ex. 208 | Ex. 209 | Ex. 210 |
| Re | 30-75% | 40-75% | 45-75% | 45-70% |
| Cr | 25-70% | 25-65% | 25-55% | 30-55% |
| Mo | 0-25% | 0-25% | 1-25% | 2-25% |
| Bi | 0-25% | 0-25% | 0-25% | 0-25% |
| Cr | 0-25% | 0-25% | 0-25% | 0-25% |
| Ir | 0-25% | 0-25% | 0-25% | 0-25% |
| Nb | 0-25% | 0-25% | 0-25% | 0-25% |
| Ta | 0-25% | 0-25% | 0-25% | 0-25% |
| V | 0-25% | 0-25% | 0-25% | 0-25% |
| W | 0-25% | 0-25% | 0-25% | 0-25% |
| Mn | 0-25% | 0-25% | 0-25% | 0-25% |
| Tc | 0-25% | 0-25% | 0-25% | 0-25% |
| Ru | 0-25% | 0-25% | 0-25% | 0-25% |
| Rh | 0-25% | 0-25% | 0-25% | 0-25% |
| Hf | 0-25% | 0-25% | 0-25% | 0-25% |
| Os | 0-25% | 0-25% | 0-25% | 0-25% |
| Cu | 0-25% | 0-25% | 0-25% | 0-25% |
| Ir | 0-25% | 0-25% | 0-25% | 0-25% |
| Ti | 0-25% | 0-25% | 0-25% | 0-25% |
| Y | 0-25% | 0-25% | 0-25% | 0-25% |
| Zr | 0-25% | 0-25% | 0-25% | 0-25% |

In Examples 1-210, it will be appreciated that all of the above ranges include any value between the range and any other range that is between the ranges set forth above. Any of the above values that include the ≤ symbol includes the range from 0 to the stated value and all values and ranges therebetween.

In accordance with another and/or alternative non-limiting aspect of the present disclosure, metal alloy used to partially or fully form the medical device can optionally 1) increase the radiopacity of the medical device, 2) increase the radial strength of the medical device, 3) increase the yield strength and/or ultimate tensile strength of the medical device, 4) improve the stress-strain properties of the medical device, 5) improve the crimping and/or expansion properties of the medical device, 6) improve the bendability and/or flexibility of the medical device, 7) improve the strength and/or durability of the medical device, 8) increase the hardness of the medical device, 9) reduce the amount of recoil of the medical device, 10) improve the biostability and/or biocompatibility properties of the medical device, 11) increase fatigue resistance of the medical device, 12) resist cracking and resist propagation of cracks of the medical device, 13) reduce or eliminate the nickel and/or chromium content of medical devices so as to reduce or eliminate problems associated with allergic reactions with surrounding tissue that can be at least partially caused by chromium and/or nickel, 14) reduce ion release of nickel and/or chromium so as to reduce or eliminate problems associated with allergic reactions with surrounding tissue that can be at least partially caused by chromium and/or nickel, 15) increased surface hardness of the medical device, 16) reduce adverse tissue reactions after implant of the medical device, 17) reduce metal ion release from the medical device after implant of the medical device, 18) reduce corrosion of the medical device after implant of the medical device, 19) reduce allergic reaction to the medical device after implant of the medical device, 20) improve hydrophilicity of the medical device, 21) reduce magnetic susceptibility of the medical device when implanted in a patient, and/or 22) reduce toxicity of the medical device after implantation of the medical device.

In accordance with another and/or alternative non-limiting aspect of the present disclosure, the medical device is optionally subjected to one or more manufacturing processes such as, but are not limited to, expansion, laser cutting, etching, crimping, annealing, drawing, pilgering, electroplating, electro-polishing, machining, plasma coating, 3D printing, 3D printing of coatings, nitriding, chemical vapor deposition, chemical polishing, cleaning, pickling, ion beam deposition or implantation, sputter coating, vacuum deposition, plasma vapor deposition, ALD, PE-CVD, etc.

In accordance with another and/or alternative non-limiting aspect of the present disclosure, the metal alloy used to partially or fully form the medical device optionally includes a certain amount of carbon, oxygen and/or nitrogen; however, this is not required. These elements have been found to affect the forming properties, brittleness and/or ductility of some metal alloys. The controlled atomic ratio of carbon, oxygen and/or nitrogen of the metal alloy also can be used to minimize the tendency of the metal alloy to form micro-cracks during the forming of the metal alloy into the medical device, and/or during the use and/or expansion of the medical device in the patient. In one non-limiting embodiment, the carbon to oxygen atomic ratio can be as low as about 0.2:1 (e.g., 0.2:1 to 50:1 and all values and ranges therebetween). In another non-limiting embodiment, the carbon content of the metal alloy can be less than about 0.2 wt. % (e.g., 0 wt. % to 0.1999999 wt. % and all values and ranges therebetween). In another non-limiting embodiment, the oxygen content of the metal alloy can be less than about 0.1 wt. % (e.g., 0 wt. to 0.0999999 wt. % and all values and ranges therebetween). In another non-limiting embodiment, the metal alloy can include less than about 0.001 wt. % nitrogen (e.g., 0 wt. % to 0.0009999 wt. % and all values and ranges therebetween). In another non-limiting embodiment, the atomic ratio of carbon to nitrogen in the metal alloy can be at least about 1.5:1 (e.g., 1.5:1 to 400:1 and all values and ranges therebetween), and the atomic ratio of oxygen to nitrogen in the metal alloy can be at least about 1.2:1 (e.g., 1.2:1 to 150:1 and all value and ranges therebetween).

In another and/or alternative non-limiting aspect of the present disclosure, the metal alloy used to form all or part of the medical device 1) is optionally not clad, metal coated, metal sprayed, plated and/or formed (e.g., cold worked, hot worked, etc.) onto another metal, or 2) does not have another metal or metal alloy metal sprayed, coated, plated, clad and/or formed onto the metal alloy.

In another and/or alternative non-limiting aspect of the present disclosure, the metal alloy used to form all or part of the medical device can be used to form a) a coating (e.g., cladding, dip coating, spray coating, plated coating, welded coating, plasma coating, etc.) on a portion of all of the medical device, or b) a core of a portion or all of the medical device. The composition of the coating, when used, can be different from the composition of the material surface to which the metal composition of the metal that is coated. The coating thickness of the metal alloy is non-limiting (e.g., 1 μm to 1 inch and all values and ranges therebetween). In one non-limiting example, the material that is to be coated is formed of chromium alloy, titanium, titanium alloy, stainless steel, iron alloy, CoCr alloy, rhenium alloy, molybdenum alloy, tungsten alloy, Ta—W alloy, refractory metal alloy, metal alloy that includes 15 awt. % rhenium, MoTa alloy, MoRe alloy, etc.), polymer material, ceramic material or composite material, and the coating layer is formed of a different material (e.g., chromium alloy, titanium, titanium alloy, stainless steel, iron alloy, CoCr alloy, rhenium alloy, molybdenum alloy, tungsten alloy, Ta—W alloy, refractory metal alloy, metal alloy that includes 15 awt. % rhenium, MoTa alloy, MoRe alloy, polymer, ceramic material, composite material, etc.). In one non-limiting example, the coating material is a metal alloy that includes at least 15 awt. % rhenium. The core or material that is coated and the outer coating layer can each form 10-99% (and all values and ranges therebetween) of the overall cross section of the coating material and coated material. When the outer metal coating is a rhenium containing alloy, such rhenium alloy can be used to create a hard surface on the medical device at specific locations as well as all over the surface. The base hardness of the rhenium containing alloy can be as low as 300 Vickers and/or as high as 500 Vickers (and all values and ranges therebetween). In instances where the properties of the fully annealed material are desired, but only the surface requires to be hardened, the present disclosure includes a method that can provide benefits of both a softer metal alloy with a harder outer surface or shell. As can be appreciated, other inner and outer hardness values can be used for the medical device.

In another non-limiting embodiment, the medical device can be partially or fully formed from a rod or tube. An inner layer of the rod (e.g., rod core, etc.) or tube can be formed of a one type of metal alloy [e.g., chromium alloy, titanium, titanium alloy, stainless steel, iron alloy, CoCr alloy, rhenium alloy, molybdenum alloy, tungsten alloy, Ta—W alloy, refractory metal alloy, MoTa alloy, MoRe alloy, etc.) and the outside of the rod or tube can be coated with one or more other materials (e.g., another type of metal or metal alloy [e.g., chromium alloy, titanium, titanium alloy, stainless steel, iron alloy, CoCr alloy, rhenium alloy, molybdenum alloy, tungsten alloy, Ta—W alloy, refractory metal alloy, MoTa alloy, MoRe alloy, etc.), polymer coating, ceramic coating, composite material coating, etc.). Non-limiting benefits of using the rhenium containing alloy in the core or interior layer of the material used to partially or fully form the medical device can include reducing the size of the medical device, increasing the strength of the medical device, and/or maintaining or reducing the cost of the medical device. As can be appreciated, the use of the rhenium containing alloy can result in other or additional advantages. The core size and/or thickness of the material used to form the core are non-limiting. The core or lower layer and the outer layer of the layered material can each form 10-99% (and all values and ranges therebetween) of the overall cross section of the layered material.

In accordance with another and/or alternative non-limiting aspect of the present disclosure, the medical device can be at least partially (e.g., 1-99.999 wt. % and all values and ranges therebetween) or fully formed from by 3D printing.

In accordance with another and/or alternative non-limiting aspect of the present disclosure, the average grain size of the metal alloy that is used to at least partially or fully form the medical device is optionally no greater than about 4 ASTM (e.g., 4 ASTM to 20 ASTM using ASTM E112 and all values and ranges therebetween, e.g., 0.35 micron to 90 micron, and all values and ranges therebetween). In another non-limiting embodiment of the disclosure, the average tensile elongation of the metal alloy used to partially or fully form the medical device is optionally at least about 25% (e.g., 25%-50% average tensile elongation and all values and ranges therebetween).

In accordance with another and/or alternative non-limiting aspect of the present disclosure, the medical device is generally designed to include at least about 5 wt. % of the metal alloy (e.g., 5-100 wt. % and all values and ranges therebetween). In one non-limiting embodiment of the disclosure, the medical device includes at least about 50 wt. % of the metal alloy. In another non-limiting embodiment of the disclosure, the medical device includes at least about 95 wt. % of the metal alloy. In one specific configuration, when the medical device includes an expandable frame, the expandable frame is formed of 50-100 wt. % (and all values and ranges therebetween) of the metal alloy, and typically 75-100 wt. % of the metal alloy.

In accordance with another and/or alternative non-limiting aspect of the present disclosure, the metal alloy can optionally be nitrided; however, this is not required. The nitrided layer on the metal alloy can function as a lubricating surface during the optional drawing of the metal alloy when partially or fully forming the medical device. After the metal alloy is nitrided, the metal alloy is typically cleaned; however, this is not required. During the nitriding process, the surface of the metal alloy is modified by the presence of nitrogen. The nitriding process can be by gas nitriding, salt bath nitriding, or plasma nitriding. In gas nitriding, the nitrogen diffuses onto the surface of the metal alloy, thereby creating a nitrided layer. The thickness and phase constitution of the resulting nitriding layers can be selected and the process optimized for the particular properties required. During gas nitriding, the metal alloy is generally nitrided in the presence of nitrogen gas or a nitrogen gas mixture (e.g., 90-99% vol. % N and 1-10 vol. % H, etc.) for at least 10 seconds at a temperature of at least about 400° C. (e.g., 400-1000° C. and all values and ranges therebetween). In one non-limiting nitriding process, the metal alloy is heated in the presence of nitrogen or a nitrogen-hydrogen mixture to a temperature of at least 400° C., and generally about 400-800° C. (and all values and ranges therebetween) for at least 10 seconds (e.g., 10 seconds to 60 minutes and all values and ranges therebetween), and generally about 1-30 minutes. In salt bath nitriding, a nitrogen-containing salt such as cyanide salt is used. During the salt bath nitriding, the metal alloy is generally exposed to temperatures of about 520-590° C. In plasma nitriding, the gas used for plasma nitriding is usually pure nitrogen. Plasma nitriding is often coupled with physical vapor deposition (PVD) process; however, this is not required. Plasma nitriding of the metal alloy generally occurs at a temperature of 220-630° C. (and all values and ranges therebetween). The metal alloy can optionally be exposed to argon and/or hydrogen gas prior to the nitriding process to clean and/or preheat the metal alloy. These gases can be optionally used to clean oxide layers and/or solvents from the surface of the metal alloy. During the nitriding process, the metal alloy can optionally be exposed to hydrogen gas to inhibit or prevent the formation of oxides on the surface of the metal alloy. The thickness of the nitrided surface layer is less than about 1 mm. In one non-limiting embodiment, the thickness of the nitride surface layer is at least about 50 nanometer and less than about 1 mm (and all values and ranges therebetween). In another non-limiting embodiment, the thickness of the nitrided surface layer is at least about 50 nanometer and less than about 0.1 mm. Generally, the weight percent of nitrogen in the nitrided surface layer is 0.0001-5 wt. % nitrogen (and all values and ranges therebetween). In one non-limiting embodiment, the weight percent of nitrogen in the nitrided surface layer is generally less than one of the primary components of the metal alloy, and typically less than each of the two primary components of the metal alloy. For example, when a metal alloy is nitrided, the weight percent of the nitrogen in the nitrided surface layer is less than a weight percent of the rhenium in the nitrided surface layer. In one non-limiting composition of the nitrided surface layer on a metal alloy (e.g., 47-55 wt. % rhenium, 10-46 wt. % molybdenum, 0.1-30 wt. % additional metal alloying agent), the nitrided surface layer comprises at least 40 wt. % rhenium, at least 8 wt. % molybdenum, and 0.0001-5 wt. % nitrogen (and all values and ranges therebetween). In one non-limiting embodiment of the disclosure, the surface of the metal alloy is nitrided prior to at least one drawing step for the metal alloy. In another non-limiting aspect of this disclosure, after the metal alloy has been annealed, the metal alloy is nitrided prior to being drawn. In another and/or alternative non-limiting embodiment, the metal alloy is cleaned to remove nitride compounds on the surface of the metal alloy prior to annealing the metal alloy. The nitride compounds can be removed by a variety of steps such as, but not limited to, grit blasting, polishing, etc. After the metal alloy has been annealed, the metal alloy can be again nitrided prior to one or more drawing steps; however, this is not required. As can be appreciated, the complete outer surface of the metal alloy can be nitrided or a portion of the outer surface of the metal alloy can be nitrided. Nitriding only selected portions of the outer surface of the metal alloy can be used to obtain different surface characteristics of the metal alloy; however, this is not required. As can be appreciated, the final formed metal alloy can include a nitrided outer surface. The nitriding process for the metal alloy can be used to increase surface hardness and/or wear resistance of the medical device, and/or to inhibit or prevent discoloration of the metal alloy (e.g., discoloration by oxidation, etc.). For example, the nitriding process can be used to increase the wear resistance of articulation surface or surfaces wear on the metal alloy used in the medical device to extend the life of the medical device, and/or increase the wear life of mating surfaces on the medical device (e.g., polyethylene liners of joint implants like knees, hips, shoulders, etc.), and/or to reduce particulate generation from use of the medical device, and/or to maintain the outer surface appearance of the metal alloy on the medical device.

In accordance with another and/or alternative non-limiting aspect of the present disclosure, the metal alloy, just prior to or after being partially or fully formed into the desired medical device, can optionally be cleaned, polished, sterilized, nitrided, etc., for final processing of the metal alloy. In one non-limiting embodiment of the disclosure, the metal alloy is electropolished. In one non-limiting aspect of this embodiment, the metal alloy is cleaned prior to being exposed to the polishing solution; however, this is not required. The cleaning process (when used) can be accomplished by a variety of techniques such as, but not limited to, 1) using a solvent (e.g., acetone, methyl alcohol, etc.) and wiping the metal alloy with a Kimwipe or other appropriate towel, and/or 2) by at least partially dipping or immersing the metal alloy in a solvent and then ultrasonically cleaning the metal alloy. As can be appreciated, the metal alloy can be cleaned in other or additional ways. In another and/or alternative non-limiting aspect of this embodiment, the polishing solution can include one or more acids. In yet another and/or alternative non-limiting aspect of this embodiment, the metal alloy is rinsed with water and/or a solvent and allowed to dry to remove polishing solution on the metal alloy.

In accordance with another and/or alternative non-limiting aspect of the present disclosure, the medical device can optionally contain and/or be coated with one or more agents that facilitate in the success of the medical device and/or treated area. The term “agent” includes, but is not limited to, a substance, pharmaceutical, biologic, veterinary product, drug, and analogs or derivatives otherwise formulated and/or designed to prevent, inhibit, and/or treat one or more clinical and/or biological events, and/or to promote healing. Non-limiting examples of clinical events that can be addressed by one or more agents include, but are not limited to, viral, fungus and/or bacterial infection; vascular diseases and/or disorders, digestive diseases and/or disorders, reproductive diseases and/or disorders, lymphatic diseases and/or disorders, cancer, implant rejection, pain, nausea, swelling, arthritis, bone diseases and/or disorders, organ failure, immunity diseases and/or disorders, cholesterol problems, blood diseases and/or disorders, lung diseases and/or disorders, heart diseases and/or disorders, brain diseases and/or disorders, neuralgia diseases and/or disorders, kidney diseases and/or disorders, ulcers, liver diseases and/or disorders, intestinal diseases and/or disorders, gallbladder diseases and/or disorders, pancreatic diseases and/or disorders, psychological disorders, respiratory diseases and/or disorders, gland diseases and/or disorders, skin diseases and/or disorders, hearing diseases and/or disorders, oral diseases and/or disorders, nasal diseases and/or disorders, eye diseases and/or disorders, fatigue, genetic diseases and/or disorders, burns, scarring and/or scars, trauma, weight diseases and/or disorders, addiction diseases and/or disorders, hair loss, cramp, muscle spasms, tissue repair, nerve repair, neural regeneration, and/or the like. The type and/or amount of agent included in the medical device and/or coated on medical device can vary. When two or more agents are included in and/or coated on the medical device, the amount of the two or more agents can be the same or different. The one or more agents can be coated on and/or impregnated in the medical device by a variety of mechanisms such as, but not limited to, spraying (e.g., atomizing spray techniques, etc.), flame spray coating, powder deposition, dip coating, flow coating, dip-spin coating, roll coating (direct and reverse), sonication, brushing, plasma deposition, depositing by vapor deposition, MEMS technology, and rotating mold deposition. In another and/or alternative non-limiting embodiment of the disclosure, the type and/or amount of agent included on, in, and/or in conjunction with the medical device is generally selected for the treatment of one or more medical treatments. The amount of two or more agents on, in, and/or used in conjunction with the medical device can be the same or different. The one or more agents, when used on and/or in the medical device, can optionally be released in a controlled manner so the area in question to be treated is provided with the desired dosage of agent over a sustained period of time. As can be appreciated, controlled release of one or more agents on the medical device is not always required and/or desirable. As such, one or more of the agents on and/or in the medical device can be uncontrollably released from the medical device during and/or after insertion of the medical device in the treatment area. It can also be appreciated that one or more agents on and/or in the medical device can be controllably released from the medical device and one or more agents on and/or in the medical device can be uncontrollably released from the medical device. It can also be appreciated that one or more agents on and/or in one region of the medical device can be controllably released from the medical device and one or more agents on and/or in the medical device can be uncontrollably released from another region on the medical device. As such, the medical device can be designed such that 1) all the agent on and/or in the medical device is controllably released, 2) some of the agent on and/or in the medical device is controllably released and some of the agent on the medical device is non-controllably released, or 3) none of the agent on and/or in the medical device is controllably released. The medical device can also be designed such that the rate of release of the one or more agents from the medical device is the same or different. The medical device can also be designed such that the rate of release of the one or more agents from one or more regions on the medical device is the same or different. Non-limiting arrangements that can be used to control the release of one or more agents from the medical device include 1) at least partially coating one or more agents with one or more polymers, 2) at least partially incorporating and/or at least partially encapsulating one or more agents into and/or with one or more polymers, and/or 3) inserting one or more agents in pores, passageway, cavities, etc., in the medical device and at least partially coat or cover such pores, passageway, cavities, etc., with one or more polymers. As can be appreciated, other or additional arrangements can be used to control the release of one or more agents from the medical device. The one or more polymers, when used to at least partially control the release of one or more agents from the medical device, can be porous or non-porous. The one or more agents can be inserted into and/or applied to one or more surface structures and/or micro-structures on the medical device, and/or be used to at least partially form one or more surface structures and/or micro-structures on the medical device. As such, the one or more agents on the medical device can be 1) coated on one or more surface regions of the medical device, 2) inserted and/or impregnated in one or more surface structures and/or micro-structures, etc., of the medical device, and/or 3) form at least a portion or be included in at least a portion of the structure of the medical device. When the one or more agents are coated on the medical device, the one or more agents can 1) be directly coated on one or more surfaces of the medical device, 2) be mixed with one or more coating polymers or other coating materials and then at least partially coated on one or more surfaces of the medical device, 3) be at least partially coated on the surface of another coating material that has been at least partially coated on the medical device, and/or 4) be at least partially encapsulated between a) a surface or region of the medical device and one or more other coating materials and/or b) two or more other coating materials. As can be appreciated, many other coating arrangements can be additionally or alternatively used. When the one or more agents are optionally inserted and/or impregnated in one or more internal structures, surface structures and/or micro-structures of the medical device, 1) one or more other coating materials can be applied at least partially over the one or more internal structures, surface structures, and/or micro-structures of the medical device, and/or 2) one or more polymers can be combined with one or more agents. As such, the one or more agents can be 1) embedded in the structure of the medical device, 2) positioned in one or more internal structures of the medical device, 3) encapsulated between two polymer coatings, 4) encapsulated between the base structure and a polymer coating, 5) mixed in the base structure of the medical device that includes at least one polymer coating, or 6) one or more combinations of 1, 2, 3, 4, and/or 5. In addition or alternatively, the one or more coating of the one or more polymers on the medical device can include 1) one or more coatings of non-porous polymers, 2) one or more coatings of a combination of one or more porous polymers and one or more non-porous polymers, 3) one or more coating of porous polymer, or 4) one or more combinations of options 1, 2, and 3. As can be appreciated, different agents can optionally be located in and/or between different polymer coating layers and/or on the structure of the medical device. As can also be appreciated, many other and/or additional coating combinations and/or configurations can be used. The concentration of one or more agents, the type of polymer, the type and/or shape of internal structures in the medical device, and/or the coating thickness of one or more agents can be used to control the release time, the release rate, and/or the dosage amount of one or more agents; however, other or additional combinations can be used. As such, the agent and polymer system combination and location on the medical device can be numerous. As can also be appreciated, one or more agents can be deposited on the top surface of the medical device to provide an initial uncontrolled burst effect of the one or more agents prior to the 1) controlled release of the one or more agents through one or more layers of a polymer system that include one or more non-porous polymers, and/or 2) uncontrolled release of the one or more agents through one or more layers of a polymer system. The one or more agents and/or polymers can be coated on the medical device by a variety of mechanisms such as, but not limited to, spraying (e.g., atomizing spray techniques, etc.), dip coating, roll coating, sonication, brushing, plasma deposition, and/or depositing by vapor deposition.

In accordance with another and/or alternative non-limiting aspect of the present disclosure, a variety of polymers can optionally be coated on the medical device and/or be used to form at least a portion of the medical device. The one or more polymers can be used on the medical device for a variety of reasons such as, but not limited to, 1) forming a portion of the medical device, 2) improving a physical property of the medical device (e.g., improve strength, improve durability, improve biocompatibility, reduce friction, etc.), 3) forming a protective coating on one or more surface structures on the medical device, 4) at least partially forming one or more surface structures on the medical device, and/or 5) at least partially controlling a release rate of one or more agents from the medical device. As can be appreciated, the one or more polymers can have other or additional uses on the medical device. The one or more polymers can be porous, non-porous, biostable, biodegradable (i.e., dissolves, degrades, is absorbed, or any combination thereof in the body), and/or biocompatible. When the medical device is coated with one or more polymers, the polymer can include 1) one or more coatings of non-porous polymers, 2) one or more coatings of a combination of one or more porous polymers and one or more non-porous polymers, 3) one or more coatings of one or more porous polymers and one or more coatings of one or more non-porous polymers, 4) one or more coating of porous polymer, or 5) one or more combinations of options 1, 2, 3, and 4. The thickness of one or more of the polymer layers can be the same or different. When one or more layers of polymer are coated onto at least a portion of the medical device, the one or more coatings can be applied by a variety of techniques such as, but not limited to, vapor deposition and/or plasma deposition, spraying, dip-coating, roll coating, sonication, atomization, brushing, and/or the like; however, other or additional coating techniques can be used. The one or more polymers that can be coated on the medical device and/or used to at least partially form the medical device can be polymers that are considered to be biodegradable, bioresorbable, or bioerodable; polymers that are considered to be biostable; and/or polymers that can be made to be biodegradable and/or bioresorbable with modification. The thickness of each polymer layer is generally at least about 0.01 μm and is generally less than about 150 μm (e.g., 0.01 μm to 150 μm and all values and ranges therebetween); however, other thicknesses can be used. In one non-limiting embodiment, the thickness of a polymer layer and/or layer of agent is about 0.02-75 μm, more particularly about 0.05-50 μm, and even more particularly about 1-30 μm. As can be appreciated, other thicknesses can be used.

In accordance with another and/or alternative non-limiting aspect of the present disclosure, the medical device, when including and/or is coated with one or more agents, can include and/or can be coated with one or more agents that are the same or different in different regions of the medical device and/or have differing amounts and/or concentrations in differing regions of the medical device. For instance, the medical device can 1) be coated with and/or include one or more biologicals on at least one portion of the medical device and at least another portion of the medical device is not coated with and/or includes agent; 2) be coated with and/or include one or more biologicals on at least one portion of the medical device that is different from one or more biologicals on at least another portion of the medical device; and/or 3) be coated with and/or include one or more biologicals at a concentration on at least one portion of the medical device that is different from the concentration of one or more biologicals on at least another portion of the medical device.

In accordance with another and/or alternative non-limiting aspect of the present disclosure, one or more portions of the medical device can optionally 1) include the same or different agents, 2) include the same or different amount of one or more agents, 3) include the same or different polymer coatings, 4) include the same or different coating thicknesses of one or more polymer coatings, 5) have one or more portions of the medical device controllably release and/or uncontrollably release one or more agents, and/or 6) have one or more portions of the medical device controllably release one or more agents and one or more portions of the medical device uncontrollably release one or more agents.

In accordance with another and/or alternative non-limiting aspect of the present disclosure, the medical device can optionally include a marker material that facilitates enabling the medical device to be properly positioned in a body passageway. The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.); and/or other types of electromagnetic waves (e.g., microwaves, visible light, infrared waves, ultraviolet waves, etc.). The marker material can form all or a portion of the medical device and/or be coated on one or more portions (flaring portion and/or body portion, at ends of medical device, at or near transition of body portion and flaring section, etc.) of the medical device. The location of the marker material can be on one or multiple locations on the medical device. The size of the one or more regions including the marker material can be the same or different. The marker material can be spaced at defined distances from one another to form ruler-like markings on the medical device to facilitate in the positioning of the medical device in a body passageway. The marker material can be a rigid or flexible material. The marker material can be a biostable or biodegradable material.

In accordance with another and/or alternative non-limiting aspect of the present disclosure, the medical device or one or more regions of the medical device can optionally be constructed by use of one or more microelectromechanical manufacturing (MEMS) techniques (e.g., micro-machining, laser micro-machining, micro-molding, etc.); however, other or additional manufacturing techniques can be used.

In accordance with another and/or alternative non-limiting aspect of the present disclosure, the medical device can optionally include one or more surface structures (e.g., pore, channel, pit, rib, slot, notch, bump, teeth, needle, well, hole, groove, etc.). These structures can be at least partially formed by MEMS (e.g., micro-machining, etc.) technology and/or other types of technology.

In accordance with another and/or alternative non-limiting aspect of the present disclosure, the medical device can optionally include one or more micro-structures (e.g., micro-needle, micro-pore, micro-cylinder, micro-cone, micro-pyramid, micro-tube, micro-parallelopiped, micro-prism, micro-hemisphere, teeth, rib, ridge, ratchet, hinge, zipper, zip-tie like structure, etc.) on the surface of the medical device. As defined herein, a “micro-structure” is a structure having at least one dimension (e.g., average width, average diameter, average height, average length, average depth, etc.) that is no more than about 2 mm, and typically no more than about 1 mm. As can be appreciated, when the medical device includes one or more surface structures, 1) all the surface structures can be micro-structures, 2) all the surface structures can be non-micro-structures, or 3) a portion of the surface structures can be micro-structures and a portion can be non-micro-structures. Typically, the micro-structures (when formed) extend from or into the outer surface no more than about 400 microns (0.01-400 microns and all values and ranges therebetween), and more typically less than about 300 microns, and more typically about 15-250 microns; however, other sizes can be used. The micro-structures can be clustered together or disbursed throughout the surface of the medical device. Similar shaped and/or sized micro-structures and/or surface structures can be used, or different shaped and/or sized micro-structures can be used. When one or more surface structures and/or micro-structures are designed to extend from the surface of the medical device, the one or more surface structures and/or micro-structures can be formed in the extended position and/or be designed to extend from the medical device during and/or after deployment of the medical device in a treatment area. The micro-structures and/or surface structures can be designed to contain and/or be fluidly connected to a passageway, cavity, etc.; however, this is not required. The one or more surface structures and/or micro-structures can be used to engage and/or penetrate surrounding tissue or organs once the medical device has been positioned on and/or in a patient; however, this is not required. The one or more surface structures and/or micro-structures can be used to facilitate in forming and maintaining a shape of a medical device. In one non-limiting embodiment, the one or more surface structures and/or micro-structures can be at least partially formed of an agent and/or be formed of a polymer. One or more of the surface structures and/or micro-structures can include one or more internal passageways that can include one or more materials (e.g., agent, polymer, etc.); however, this is not required. The one or more coatings and/or one or more surface structures and/or micro-structures of the medical device can be used for a variety of purposes such as, but not limited to, 1) increasing the bonding and/or adhesion of one or more agents, adhesives, marker materials, and/or polymers to the medical device, 2) changing the appearance or surface characteristics of the medical device, and/or 3) controlling the release rate of one or more agents. The one or more micro-structures and/or surface structures can be biostable, biodegradable, etc. The medical device or one or more regions of the medical device can be at least partially covered and/or filled with a protective material to at least partially protect one or more regions of the medical device, and/or one or more micro-structures, and/or surface structures on the medical device from damage. The protective material can include one or more polymers previously identified above. The protective material can be 1) biostable and/or biodegradable and/or 2) porous and/or non-porous.

In accordance with another and/or alternative non-limiting aspect of the present disclosure, the medical device can optionally be an expandable device that can be expanded by use of some other device (e.g., balloon, etc.). The expandable medical device can be fabricated from a material that has no or substantially no shape-memory characteristics.

In accordance with another and/or alternative non-limiting aspect of the present disclosure, there is optionally provided a near net process for a frame or other metal component of the medical device. In one non-limiting embodiment of the disclosure, there is provided a method of powder pressing materials and increasing the strength post-sintering by imparting additional cold work. In one non-limiting embodiment, the green part is pressed and then sintered. Thereafter, the sintered part is again pressed to increase its mechanical strength by imparting cold work into the pressed and sintered part. Generally, the temperature during the pressing process after the sintering process is 20-100° C. (and all values and ranges therebetween), typically 20-80° C., and more typically 20-40° C. As defined herein, cold working occurs at a temperature of no more than 150° C. (e.g., 10-150° C. and all values and ranges therebetween). The change in the shape of the repressed post-sintered part needs to be determined so the final part (pressed, sintered and re-pressed) meets the dimensional requirements of the final formed part. For a metal alloy, a prepress pressure of 1-300 tsi (1 ton per square inch) (and all values and ranges therebetween) can be used followed by a sintering process of at least 1600° C. (e.g., 1600-2600° C. and all values and ranges therebetween) and a post sintering press at a pressure of 1-300 tsi (and all values and ranges therebetween) at a temperature of at least 20° C. (e.g., 20-100° C. and all values and ranges therebetween; 20-40° C., etc.). There is also provided a process of increasing the mechanical strength of a pressed metal part by repressing the post-sintered part to add additional cold work into the material, thereby increasing its mechanical strength. There is also provided a process of powder pressing to a near net or final part using metal powder. In one non-limiting embodiment, the metal powder used to form the near net or final part includes a minimum of 40 wt. % rhenium and at least 25 wt. % molybdenum, and the remainder can optionally include one or more elements of tungsten, tantalum, chromium, niobium, zirconium, iridium, titanium, bismuth, and yttrium. In another non-limiting embodiment, the metal powder used to form the near net or final part includes 40-60 wt. % rhenium (and all values and ranges therebetween), and two or more elements of tungsten, tantalum, molybdenum, chromium, niobium, zirconium, iridium, titanium, bismuth, and yttrium. In another non-limiting embodiment, the metal powder used to form the near net or final part includes 40-60 wt. % rhenium (and all values and ranges therebetween), and three or more elements of tungsten, tantalum, molybdenum, chromium, niobium, zirconium, iridium, titanium, bismuth, and yttrium. In another non-limiting embodiment, the metal powder used to form the near net or final part includes 41-58.5 wt. % rhenium (and all values and ranges therebetween), and 18-45 wt. % molybdenum (and all values and ranges therebetween), and one or more additional elements of tantalum, chromium, niobium, zirconium, iridium, titanium, bismuth, and yttrium. In another non-limiting embodiment, the metal powder used to form the near net or final part includes 41-58.5 wt. % rhenium (and all values and ranges therebetween), and 18-45 wt. % molybdenum (and all values and ranges therebetween), and two or more additional elements of tantalum, chromium, niobium, zirconium, iridium, titanium, bismuth, and yttrium. In another non-limiting embodiment, the metal powder used to form the near net or final part includes 41-58.5 wt. % rhenium (and all values and ranges therebetween), and 18-45 wt. % molybdenum (and all values and ranges therebetween), and one or more elements of tantalum, chromium, niobium, and zirconium, wherein the combined weight percent of the one of more additional elements is 11-41 wt. %. In another non-limiting embodiment, the metal powder used to form the near net or final part includes 41-58.5 wt. % rhenium (and all values and ranges therebetween), and 18-45 wt. % molybdenum (and all values and ranges therebetween), and two or more elements of tantalum, chromium, niobium, and zirconium, wherein the combined weight percent of the two of more additional elements is 11-41 wt. %.

In accordance with another and/or alternative non-limiting aspect of the present disclosure, there is optionally provided a press of near net or finished part composite. The process of pressing metals into near net of finished parts is well established; however, pressing a composite structure formed of metal powder and polymer for purposes of making complex part geometries and foam-like structures is new. Similarly, using a pressing process to impart particular biologic substances into the metal matrix is also new. In one non-limiting embodiment, there is provided a process of creating a metal part with pre-defined voids to create a trabecular or foam structure composed of mixing a metal and polymer powder, pressing the powder into a finished part or semi-finished green part, and then sintering the part under which conditions the polymer leaves the metal behind through a process of thermal degradation of the polymer. The resulting part has a porosity associated with the size of the polymer particles as well as the homogeneity of the mixture upon pressing prior to sintering. In another non-limiting embodiment, there is provided a process by which a residual of the polymer is left behind after thermal degradation, on the metal substrate, and the polymer residual has some desired biological affect (e.g., masking the metal from the body by encapsulation, promotion of cellular attachment and growth). The polymer and metal powders can be of varying sizes to create multiple voids—some large to create a pathway for cellular growth, and some small to create a ruff surface to promote cellular attachment. As can be appreciated, the polymer can be uniformly or non-uniformly dispersed with the metal powder. For example, if the final formed part is to have a uniform density and pore structure, the polymer material is uniformly dispersed with the metal powder prior to consolidating and pressing the polymer and metal powders together and then subsequently sintering together the metal powder to form the metal part or medical device. Alternatively, if the formed metal part or medical device is to have one or more channels, passageways, and/or voids on the outer surface and/or within the formed part or medical device, at least a portion of the polymer is not uniformly distributed with the metal powder, but instead is concentrated or forms all of the region that is to be the one or more channels, passageways, and/or voids on the outer surface and/or within the formed part or medical device such that when the polymer and metal powder is sintered, some or all of the polymer is degraded and removed from the part or medical device, thereby forming such one or more channels, passageways, and/or voids on the outer surface and/or within the formed part or medical device. As such, the use of the polymer in combination with metal powder and subsequent pressing and sintering can be used to form novel and customized shapes for the medical device or the near net form of the medical device. Generally, the polymer constitutes about 0.1-70 vol. % (and all values and ranges therebetween) of the consolidated and pressed material prior to the sintering step, typically the polymer constitutes about 1-60 vol. % of the consolidated and pressed material prior to the sintering step, more typically the polymer constitutes about 2-50 vol. % of the consolidated and pressed material prior to the sintering step, and even more typically the polymer constitutes about 2-45 vol. % of the consolidated and pressed material prior to the sintering step. As such, if the polymer constitutes about 5 vol. % of the consolidated and pressed material prior to the sintering step, if after the sintering step at least 99% of the polymer is degraded and removed from the part or medical device, then the part could include up to about 5 vol. % cavities and/or passageways in the part or medical device. The type of polymer and the type of metal powder is non-limiting. The polymer and metal powders can be of varying sizes to create multiple voids/passageways/channels which can be used to create a pathway for cellular growth, create a ruff surface to promote cellular attachment, have a biological agent inserted into one or more of the voids/passageways/channels, have biological material inserted into one or more of the voids/passageways/channels, etc. In one non-limiting embodiment, the average particle size of the polymer is greater than the average particle size of the metal powder.

In accordance with another and/or alternative non-limiting aspect of the present disclosure, after the sintering process, at least 98 vol. % of the polymer is thermally degraded and/or removed from the sintered material, typically at least 99 vol. % of the polymer is thermally degraded and/or removed from the sintered material, more typically at least 99.5 vol. % of the polymer is thermally degraded and/or removed from the sintered material, still even more typically at least 99.9 vol. % of the polymer is thermally degraded and/or removed from the sintered material, and even still more typically at least 99.95 vol. % of the polymer is thermally degraded and/or removed from the sintered material. The resulting part or medical device has a porosity associated with the size of the polymer particles as well as the homogeneity of the mixture upon pressing prior to sintering.

In accordance with another and/or alternative non-limiting aspect of the present disclosure, after the sintering process, some of the polymer remains in the sintered part of the medical device. The remaining polymer in the sintered part of the medical device can optionally have some desired biological affect (e.g., masking the metal from the body by encapsulation, promotion of cellular attachment and growth). The remaining polymer can optionally include one or more biological agents that remain active after the sintering process. In one non-limiting embodiment, after the sintering process, about 5-97.5 vol. % (and all values and ranges therebetween) of the polymer is thermally degraded and/or removed from the sintered material, typically about 10-95 vol. % of the polymer is thermally degraded and removed from the sintered material, and more typically about 10-80 vol. % of the polymer is thermally degraded and removed from the sintered material.

In accordance with another and/or alternative non-limiting aspect of the present disclosure, the metal alloy used to at least partially form the medical device is initially formed into a blank, a rod, a tube, etc., and then finished into final form by one or more finishing processes. The metal alloy blank, rod, tube, etc., can be formed by various techniques such as, but not limited to, 1) melting the metal alloy and/or metals that form the metal alloy (e.g., vacuum arc melting, etc.) and then extruding and/or casting the metal alloy into a blank, rod, tube, etc., 2) melting the metal alloy and/or metals that form the metal alloy, forming a metal strip, and then rolling and welding the strip into a blank, rod, tube, etc., or 3) consolidating the metal powder of the metal alloy and/or metal powder of metals that form the metal alloy into a blank, rod, tube, etc. When the metal alloy is formed into a blank, the shape and size of the blank is non-limiting. In one non-limiting process, the near net medical device, blank, rod, tube, etc., can be formed from one or more ingots of metal or metal alloy. In one non-limiting process, an arc melting process (e.g., vacuum arc melting process, etc.) can be used to form the near net medical device, blank, rod, tube, etc. In another non-limiting process, rhenium powder and tungsten powder and optionally molybdenum powder can be placed in a crucible (e.g., silica crucible, etc.) and heated under a controlled atmosphere (e.g., vacuum environment, carbon monoxide environment, hydrogen and argon environment, helium, argon, etc.) by an induction melting furnace to form the near net medical device, blank, rod, tube, etc. It can be appreciated that other or additional processes can be used to form the metal alloy. In one non-limiting embodiment, the average particle size of the metal powders is less than about 230 mesh (e.g., less than 63 microns). In another and/or alternative non-limiting embodiment, the average particle size of the metal powders is about 2-63 microns, and more particularly about 5-40 microns. As can be appreciated, smaller average particle sizes can be used. The purity of the metal powders should be selected so that the metal powders contain very low levels of carbon, oxygen, and nitrogen. Typically, the carbon content of the metal powder used to form the metal alloy is less than about 100 ppm, the oxygen content is less than about 50 ppm, and the nitrogen content is less than about 20 ppm. Typically, metal powder used to form the metal alloy has a purity grade of at least 99.9 and more typically at least about 99.95. The blend of metal powder is then pressed together to form a solid solution of the metal alloy into a near net medical device, blank, rod, tube, etc. Typically, the pressing process is by an isostatic process (i.e., uniform pressure applied from all sides on the metal powder); however other processes can be used. When the metal powders are pressed together isostatically, cold isostatic pressing (CIP) is typically used to consolidate the metal powders; however, this is not required. The pressing process can be performed in an inert atmosphere, an oxygen-reducing atmosphere (e.g., hydrogen, argon and hydrogen mixture, etc.), and/or under a vacuum; however, this is not required. The average density of the near net medical device, blank, rod, tube, etc., that is achieved by pressing together the metal powders is about 80-95% (and all values and ranges therebetween) of the final average density of the near net medical device, blank, rod, tube, etc., or about 70-96% (and all values and ranges therebetween) the minimum theoretical density of the metal alloy. Pressing pressures of at least about 300 MPa are generally used. Generally, the pressing pressure is about 400-700 MPa; however, other pressures can be used. After the metal powders are pressed together, the pressed metal powders are sintered at a temperature of at least 1600° C. (e.g., 1600-3500° C. and all values and ranges therebetween) to partially or fully fuse the metal powders together to form the near net medical device, blank, rod, tube, etc. The sintering of the consolidated metal powder can be performed in an oxygen-reducing atmosphere (e.g., helium, argon, hydrogen, argon and hydrogen mixture, etc.), and/or under a vacuum; however, this is not required. At the high sintering temperatures, a high hydrogen atmosphere will reduce both the amount of carbon and oxygen in the formed near net medical device, blank, rod, tube, etc. The sintered metal powder generally has an as-sintered average density of about 90-99% the minimum theoretical density of the metal alloy. Typically, the sintered metal alloy has a final average density of at least about 5 gm/cc, and typically at least about 8.3 gm/cc, and can be up to or greater than about 16 gm/cc; however, this is not required. The density of the formed near net medical device, blank, rod, tube, etc., will generally depend on the type of metal alloy used.

In accordance with another and/or alternative non-limiting aspect of the present disclosure, the near net medical device, blank, rod, tube, etc., can optionally be cleaned and/or polished after the near net medical device, blank, rod, tube, etc., has been form; however, this is not required. Typically, the near net medical device, blank, rod, tube, etc., is cleaned and/or polished prior to being further processed; however, this is not required. When the near net medical device, blank, rod, tube, etc., is resized and/or annealed, the resized and/or annealed, the near net medical device, blank, rod, tube, etc., is typically cleaned and/or polished prior to and/or after each or after a series of resizing and/or annealing processes; however, this is not required. The cleaning and/or polishing of the near net medical device, blank, rod, tube, etc., is used to remove impurities and/or contaminants from the surfaces of the near net medical device, blank, rod, tube, etc. Impurities and contaminants can become incorporated into the metal alloy during the processing of the near net medical device, blank, rod, tube, etc. The inadvertent incorporation of impurities and contaminants in the near net medical device, blank, rod, tube, etc., can result in an undesired amount of carbon, nitrogen and/or oxygen, and/or other impurities in the metal alloy. The inclusion of impurities and contaminants in the metal alloy can result in premature micro-cracking of the metal alloy and/or an adverse effect on one or more physical properties of the metal alloy (e.g., decrease in tensile elongation, increased ductility, increased brittleness, etc.). The cleaning of the metal alloy can be accomplished by a variety of techniques such as, but not limited to, 1) using a solvent (e.g., acetone, methyl alcohol, etc.) and wiping the metal alloy with a Kimwipe or other appropriate towel, 2) by at least partially dipping or immersing the metal alloy in a solvent and then ultrasonically cleaning the metal alloy, and/or 3) by at least partially dipping or immersing the metal alloy in a pickling solution. As can be appreciated, the metal alloy can be cleaned in other or additional ways. If the metal alloy is to be polished, the metal alloy is generally polished by use of a polishing solution that typically includes an acid solution; however, this is not required.

In accordance with another and/or alternative non-limiting aspect of the present disclosure, the near net medical device, blank, rod, tube, etc., can be resized to the desired dimension of the medical device. In one non-limiting embodiment, the cross-sectional area or diameter of the near net medical device, blank, rod, tube, etc., is reduced to a final near net medical device, blank, rod, tube, etc. dimension in a single step or by a series of steps. The reduction of the outer cross-sectional area or diameter of the near net medical device, blank, rod, tube, etc., may be obtained by centerless grinding, turning, electropolishing, drawing process, grinding, laser cutting, shaving, polishing, EDM cutting, etc. The outer cross-sectional area or diameter size of the near net medical device, blank, rod, tube, etc., can be reduced by the use of one or more drawing processes; however, this is not required. During the drawing process, care should be taken to not form micro-cracks in the near net medical device, blank, rod, tube, etc., during the reduction of the near net medical device, blank, rod, tube, etc., outer cross-sectional area or diameter.

In accordance with another and/or alternative non-limiting aspect of the present disclosure, there is provide a medical device that includes a coating of a) zirconium salt (e.g., Zirconium nitrate, Oxalic acid zirconium salt, AgZrPSi, AgZrP, Zirconium Phosphate-Glycinediphosphonate and silver, Zirconium Phosphate-Glycinediphosphonate and zinc, Ag_{0.2}H_{1.2}Zr_{1.9}(PO₄)₃·0.05H₂O, Ag_{0.1}H_{1.14}Zr_{1.94}(PO₄)₃·0.15H₂O, Ag_{0.2}Na_{0.4}(NH₄)_{0.84}Zr_{1.89}(PO₄)₃·0.3H₂O, Ag_{0.3}Na_{0.1}H_{1.04}Zr_{1.89}(PO₄)₃·0.2H₂O, Ag_{0.5}Na_{0.2}H_{0.3}(NH₄)_{0.32}Zr_{1.92}(PO₄)₃·0.2H₂O, Ag_{0.4}K_{0.6}H_{0.36}Zr_{1.91}(PO₄)₃·0.1H₂O, etc.) b) zirconia, and/or c) Zr(IV) complexes with amino acid or ligand (e.g., N,O bidentate ligands [e.g., C₂₀H₁₄Cl₂N₄O₆Zr; C₂₂H₁₈Cl₂N₄O₆Zr; C₂₆H₂₆Cl₂N₄O₆S₂Zr, C₂₆H₂₄Cl₄N₄O₆Zr, C₃₄H₂₆Cl₂N₄O₆Zr, C₂₂H₁₆Cl₄N₄O₆Zr, C₂₆H₂₄Cl₄N₄O₆Zr, C₃₄H₂₆Cl₂N₄O₆Zr, C₃₈H₂₈Cl₂N₆O₆Zr, etc.], Zr-amino acid complexes [e.g., Zr-glutamic acid-graphite oxide, Zr-aspartic acid-graphite oxide]). The coating at least partially includes a) zirconium salt, b) zirconia, and/or c) Zr(IV) complexes with amino acid or ligand can be used to partially or fully coat the medical device. In one non limiting embodiment, the coating at least partially includes a) zirconium salt, b) zirconia, and/or c) Zr(IV) complexes with amino acid or ligand can be applied to the medical device prior to the packaging of the medical device. The coating material can optionally be used to inhibit or prevent microbial growth on the medical device after the medical device has been packaged. In another non limiting embodiment, the coating at least partially includes a) zirconium salt, b) zirconia, and/or c) Zr(IV) complexes with amino acid or ligand can be applied to the medical device to disinfect the medical device prior to using (e.g., implanting, etc.) the medical device on the patient. For example, the medical device can be sprayed with or dipped in a solution that includes a) zirconium salt, b) zirconia, and/or c) Zr(IV) complexes with amino acid or ligand to disinfect the medical device and thereafter optionally rinse (e.g., using distilled water, etc.) the solution off the medical device prior to using the medical device in the patient.

In accordance with another and/or alternative non-limiting aspect of the present disclosure, there is provide a medical device that includes a coating that at least partially includes zirconium salt (e.g., zirconium nitrate, etc.). The zirconium salt can be the sole antimicrobial agent applied to the medical device, or the zirconium salt can be combined with one or more other antimicrobial agents when applied to the medical device. In one non-limiting embodiment, the zirconium salt forms 30-100 wt. % (and all values and ranges therebetween) of the antimicrobial coating on the medical device. The zirconium salt can be applied to the medical device via a liquid solution (e.g., solvent solution, water solution, etc.) and thereafter allow the solution to dry on the surface of the medical device. The antimicrobial coating can optionally be removed (e.g., washed off, etc.) prior to the medical device being inserted into a patient. The washing process to remove the zirconium salt, when used, can includes the use of water (e.g., sterilized water, etc.), a saline solution, acid solution (e.g., nitric acid solution, glacial acetic acid solution, etc.), etc. The thickness of the antimicrobial coating in generally at least 0.01 μm and generally 0.01-500 μm (and all values and ranges therebetween). The concentration of the zirconium salt when applied to the medical device is generally at least 5.7 μg/mL (e.g., 5.7 μg/mL to 200 μg/mL and all values and ranges therebetween). In one non-limiting embodiment, the concentration of the zirconium salt when applied to the medical device is at least 15 μg/mL. In another non-limiting embodiment, the concentration of the zirconium salt when applied to the medical device is at least 22.8 μg/mL.

In accordance with another and/or alternative non-limiting aspect of the present disclosure, there is provide a medical device that includes a coating of zirconia. The particle size of the zirconia is generally at least 0.001 μm (1 nanometer), and typically 0.001 μm to 0.5 μm (and all values and ranges therebetween). The zirconia in the coating can be the sole antimicrobial agent applied to the medical device, or the zirconia can be combined with one or more other antimicrobial agents when applied to the medical device. In one non-limiting embodiment, the zirconia forms 30-100 wt. % (and all values and ranges therebetween) of the antimicrobial coating on the medical device. The zirconia can be applied to the medical device via a liquid solution (e.g., solvent solution, water solution, etc.) and thereafter allow the solution to dry on the surface of the medical device. The antimicrobial coating can optionally be removed (e.g., washed off, etc.) prior to the medical device being inserted into a patient. The washing process to remove the zirconia, when used, can includes the use of water (e.g., sterilized water, etc.), a saline solution, acid solution (e.g., nitric acid solution, glacial acetic acid solution, etc.), etc. The thickness of the antimicrobial coating in generally at least 0.01 μm and generally 0.01-500 μm (and all values and ranges therebetween). The concentration of the zirconia when applied to the medical device is generally at least 5.7 μg/mL (e.g., 5.7 μg/mL to 200 μg/mL and all values and ranges therebetween). In one non-limiting embodiment, the concentration of the zirconia when applied to the medical device is at least 15 μg/mL. In another non-limiting embodiment, the concentration of the zirconia when applied to the medical device is at least 22.8 μg/mL.

In accordance with another and/or alternative non-limiting aspect of the present disclosure, there is provide a medical device that includes a coating of an antimicrobial agent, wherein the antimicrobial agent at least partially includes Zr(IV) complexes with amino acid or ligand (e.g., Zr-amino acid complexes, etc.). The Zr(IV) complexes with amino acid or ligand can be the sole antimicrobial agent applied to the medical device, or the Zr(IV) complexes with amino acid or ligand can be combined with one or more other antimicrobial agents when applied to the medical device. In one non-limiting embodiment, the Zr(IV) complexes with amino acid or ligand forms 30-100 wt. % (and all values and ranges therebetween) of the antimicrobial coating on the medical device. The Zr(IV) complexes with amino acid or ligand can be applied to the medical device via a liquid solution (e.g., solvent solution, water solution, etc.) and thereafter allow the solution to dry on the surface of the medical device. The antimicrobial coating can optionally be removed (e.g., washed off, etc.) prior to the medical device being inserted into a patient. The washing process to remove the Zr(IV) complexes, when used, can includes the use of water (e.g., sterilized water, etc.), a saline solution, acid solution (e.g., nitric acid solution, glacial acetic acid solution, etc.), etc. The thickness of the antimicrobial coating in generally at least 0.01 μm and generally 0.01-500 μm (and all values and ranges therebetween). The concentration of the Zr(IV) complexes with amino acid or ligand when applied to the medical device is generally at least 5.7 μg/mL (e.g., 5.7 μg/mL to 200 μg/mL and all values and ranges therebetween). In one non-limiting embodiment, the concentration of the Zr(IV) complexes with amino acid or ligand when applied to the medical device is at least 15 μg/mL. In another non-limiting embodiment, the concentration of the Zr(IV) complexes with amino acid or ligand when applied to the medical device is at least 22.8 μg/mL.

In accordance with another and/or alternative non-limiting aspect of the present disclosure, there is provide a medical device that can be form by one or more manufacturing processes. These manufacturing processes can include, but are not limited to, laser cutting, etching, annealing, drawing, pilgering, electroplating, electro-polishing, machining, plasma coating, 3D printed coatings, 3D printing, chemical vapor deposition, chemical polishing, cleaning, pickling, ion beam deposition or implantation, sputter coating, vacuum deposition, etc. In one non-limiting embodiment, at least a portion or all of the medical device is formed by a 3D printing process.

In accordance with another and/or alternative non-limiting aspect of the present disclosure, there is provide a medical device in the form of a prosthetic heart valve. The prosthetic heart valve includes an expandable frame, one or more leaflets, and optionally one or more skirts. A non-limiting prosthetic heart valve that includes an expandable frame, one or more leaflets, and one or more skirts is disclosed in U.S. Ser. No. 18/400,781 filed Dec. 29, 2023, which is fully incorporated herein by reference. The zirconium containing coating can be coated on the outer surface of one or more components of the prosthetic heart valve. In one non-limiting embodiment, the zirconium containing coating is partially (e.g., 0.1-99.999% of the outer surface and all values and ranges therebetween) or fully coated on the outer surface of the expandable frame, the one or more leaflets, and/or the one or more skirts. In another non-limiting embodiment, the zirconium containing coating is partially or fully coated on the outer surface of only the one or more leaflets, and/or the one or more skirts, and 0-5% (and all values and ranges therebetween) of the outer surface of the expandable frame is coated with the zirconium containing coating. In another non-limiting embodiment, the zirconium containing coating is partially or fully coated on the outer surface of one or more of the leaflets prior to the one or more leaflets being connected to the expandable frame of the prosthetic heart valve. In another non-limiting embodiment, the zirconium containing coating is partially or fully coated on the outer surface of one or more of the leaflets after to the one or more leaflets are connected to the expandable frame of the prosthetic heart valve. In another non-limiting embodiment, the zirconium containing coating is partially or fully coated on the outer surface of one or more of the skirts prior to the one or more skirts being connected to the expandable frame of the prosthetic heart valve. In another non-limiting embodiment, the zirconium containing coating is partially or fully coated on the outer surface of one or more of the skirts after the one or more skirts are connected to the expandable frame of the prosthetic heart valve. In another non-limiting embodiment, the zirconium containing coating is partially or fully coated on the outer surface of expandable frame prior to the one or more leaflets and/or one or more skirts are connected to the expandable frame of the prosthetic heart valve. In another non-limiting embodiment, the zirconium containing coating is partially or fully coated on the outer surface of expandable frame after the one or more leaflets and/or one or more skirts are connected to the expandable frame of the prosthetic heart valve. The zirconium containing coating can be used to inhibit or prevent microbial growth on the outer surface of one or more components of the prosthetic heart valve prior to the prosthetic heart valve being inserted into a patient. This feature of the zirconium containing coating can be beneficial in inhibiting or preventing contamination (e.g., microbial growth, dirt and/or other foreign materials on the outer surface, etc.) of the leaflets and/or skirts prior to and/or after the leaflets and/or skirts are secured to the expandable frame of the prosthetic heart valve. Prior to the insertion of the prosthetic heart valve into the body of the patient, one or more portions or all of the prosthetic heart valve can optionally be treated (e.g., washed, etc.) to partially or fully remove the zirconium containing coating for the outer surface of the one or more components or portions of the prosthetic heart valve.

One non-limiting object of the present disclosure is the provision of a medical device that is partially or fully coated with an antimicrobial agent that at least partially includes a) zirconium salt, b) zirconia, and/or c) Zr(IV) complexes with amino acid or ligand.

Another and/or alternative non-limiting object of the present disclosure is the provision of a medical device that is partially or fully formed of the metal alloy that includes at least 15 atw. % rhenium.

Another and/or alternative non-limiting object of the present disclosure is the provision of a medical device wherein a portion or all of the medical device includes a refractory metal alloy or a metal alloy that includes at least 5 awt. % (e.g., 5-99 awt. % and all values and ranges therebetween) rhenium.

Another and/or alternative non-limiting object of the present disclosure is the provision of a medical device that has improved procedural success rates.

Another and/or alternative non-limiting object of the present disclosure is the provision of an medical device that is formed of a base material and a zirconium containing coating; the base material includes a metal selected from the group consisting of a) standard stainless steel, b) standard cobalt-chromium alloy, c) standard titanium-aluminum-vanadium alloy, d) standard aluminum alloy, e) standard nickel alloy, f) standard titanium alloy, g) standard tungsten alloy, h) standard molybdenum alloy, i) standard copper alloy, j) standard beryllium-copper alloy, k) standard titanium-nickel alloy, 1) refractory metal alloy, or m) metal alloy that includes at least metal alloy that includes at least 5 atomic weight percent (awt. %) or atomic percent (awt %) rhenium (e.g., 5-99 awt. % rhenium and all values and ranges therebetween); the medical device is optionally a) a spinal implant, b) a frame and other structure for use with a spinal implant, c) a bone implant, d) an artificial disk, e) an artificial spinal disk, f) a spinal interbody, g) an expandable spinal interbody, h) an interbody fusion device, i) an expandable interbody fusion device, j) a prosthetic implant or device to repair, replace and/or support a bone and/or cartilage, k) a bone plate nail, 1) a spinal rod, m) a bone screw, n) a post, o) a spinal cage, p) a bone plate, q) a pedicle screw, r) a cap, s) a hinge, t) a joint system, u) an anchor, v) a spacer, w) a shaft, x) an anchor, y) a disk, z) a ball, aa) a tension band, or ab) a locking connector or other structural assembly that is used in a body to support a structure, mount a structure, and/or repair a structure in a body.

Another and/or alternative non-limiting object of the present disclosure is the provision of a medical device wherein the medical device includes no more than 0.1 wt. % nickel, no more than 0.1 wt. % cobalt, and/or no more than 0.1 wt. % chromium.

Another and/or alternative non-limiting object of the present disclosure is the provision of a medical device wherein the medical device is formed of the refractory metal alloy or the metal alloy that includes at least metal alloy that includes at least 5 atomic weight percent (awt. %) or atomic percent (awt %) rhenium (e.g., 5-99 awt. % rhenium and all values and ranges therebetween).

Another and/or alternative non-limiting object of the present disclosure is the provision of a medical device wherein the medical device is formed of at least 0.1 wt. % rhenium and one or more metal selected from molybdenum, chromium, cobalt, nickel, titanium, tantalum, niobium, zirconium, and/or tungsten.

Another and/or alternative non-limiting object of the present disclosure is the provision of an medical device that is formed of a base material and a zirconium containing coating; the medical device is at least partially formed from a metal selected from a metal selected from the group consisting of a) standard stainless steel, b) standard cobalt-chromium alloy, c) standard titanium-aluminum-vanadium alloy, d) standard aluminum alloy, e) standard nickel alloy, f) standard titanium alloy, g) standard tungsten alloy, h) standard molybdenum alloy, i) standard copper alloy, j) standard beryllium-copper alloy, k) standard titanium-nickel alloy, 1) refractory metal alloy, or m) metal alloy that includes at least metal alloy that includes at least 5 atomic weight percent (awt. %) or atomic percent (awt %) rhenium (e.g., 5-99 awt. % rhenium and all values and ranges therebetween); and wherein the medical device optionally includes no more than 0.1 wt. % nickel, no more than 0.1 wt. % cobalt, and/or no more than 0.1 wt. % chromium, and wherein the medical device is optionally formed of the refractory metal alloy or the metal alloy that includes at least metal alloy that includes at least 5 atomic weight percent (awt. %) or atomic percent (awt %) rhenium (e.g., 5-99 awt. % rhenium and all values and ranges therebetween), and wherein the medical device is optionally formed of at least 0.1 wt. % rhenium and one or more metal selected from molybdenum, chromium, cobalt, nickel, titanium, tantalum, niobium, zirconium, and/or tungsten, and wherein the medical device is optionally a) a spinal implant, b) a frame and other structure for use with a spinal implant, c) a bone implant, d) an artificial disk, e) an artificial spinal disk, f) a spinal interbody, g) an expandable spinal interbody, h) an interbody fusion device, i) an expandable interbody fusion device, j) a prosthetic implant or device to repair, replace and/or support a bone and/or cartilage, k) a bone plate nail, l) a spinal rod, m) a bone screw, n) a post, o) a spinal cage, p) a bone plate, q) a pedicle screw, r) a cap, s) a hinge, t) a joint system, u) an anchor, v) a spacer, w) a shaft, x) an anchor, y) a disk, z) a ball, aa) a tension band, or ab) a locking connector or other structural assembly that is used in a body to support a structure, mount a structure, and/or repair a structure in a body.

Another and/or alternative non-limiting object of the present disclosure is the provision of a medical device that is formed of a base material and a zirconium containing coating, and wherein the coating includes a) zirconium salt, b) zirconia, and/or c) Zr(IV) complexes.

Another and/or alternative non-limiting object of the present disclosure is the provision of a medical device that is formed of a base material and a zirconium containing coating, and wherein the coating has a thickness of 0.01-500 μm.

Another and/or alternative non-limiting object of the present disclosure is the provision of a medical device that is formed of a base material and a zirconium containing coating, and wherein the coating includes 30-100 wt. % of a) zirconium salt, b) zirconia, and/or c) Zr(IV) complexes.

Another and/or alternative non-limiting object of the present disclosure is the provision of a medical device that is formed of a base material and a zirconium containing coating, and wherein the coating includes zirconium salt.

Another and/or alternative non-limiting object of the present disclosure is the provision of an medical device that is formed of a base material and a zirconium containing coating, and wherein the coating includes zirconium salt, and wherein said zirconium salt includes one or more of zirconium nitrate, oxalic acid zirconium salt, AgZrPSi, AgZrP, zirconium phosphate-glycinediphosphonate and silver, zirconium phosphate-glycinediphosphonate and zinc, Ag_{0.2}H_{1.2}Zr_{1.9}(PO₄)₃·0.05H₂O, Ag_{0.1}H_{1.14}Zr_{1.94}(PO₄)₃·0.15H₂O, Ag_{0.2}Na_{0.4}(NH₄)_{0.84}Zr_{1.89}(PO₄)₃·0.3H₂O, Ag_{0.3}Na_{0.1}H_{1.04}Zr_{1.89}(PO₄)₃·0.2H₂O, Ag_{0.5}Na_{0.2}H_{0.3}(NH₄)_{0.32}Zr_{1.92}(PO₄)₃·0.2H₂O, and Ag_{0.4}K_{0.6}H_{0.36}Zr_{1.91}(PO₄)₃·0.1H₂O.

Another and/or alternative non-limiting object of the present disclosure is the provision of a medical device that is formed of a base material and a zirconium containing coating, and wherein the coating includes zirconia.

Another and/or alternative non-limiting object of the present disclosure is the provision of a medical device that is formed of a base material and a zirconium containing coating, and wherein the coating includes zirconia, and wherein said zirconia has a particle size of 0.001 μm to 0.5 μm.

Another and/or alternative non-limiting object of the present disclosure is the provision of a medical device that is formed of a base material and a zirconium containing coating, and wherein the coating includes Zr(IV) complexes.

Another and/or alternative non-limiting object of the present disclosure is the provision of a medical device that is formed of a base material and a zirconium containing coating, and wherein the coating includes Zr(IV) complexes, and wherein said Zr(IV) complexes are formed with amino acid or ligand.

Another and/or alternative non-limiting object of the present disclosure is the provision of a medical device that is formed of a base material and a zirconium containing coating, and wherein the coating includes a) zirconium salt, b) zirconia, and/or c) Zr(IV) complexes is applied to said medical device in a concentration of at least 5.7 μg/mL.

Another and/or alternative non-limiting object of the present disclosure is the provision of a medical device that is formed of a base material and a zirconium containing coating, and wherein the coating includes a) zirconium salt, b) zirconia, and/or c) Zr(IV) complexes is applied to said medical device in a concentration of 5.7 μg/mL to 200 μg/mL.

Another and/or alternative non-limiting object of the present disclosure is the provision of a medical device that is formed of a base material and a zirconium containing coating, and wherein the coating includes Zr(IV) complexes, and wherein said Zr(IV) complexes include one or more of C₂₀H₁₄Cl₂N₄O₆Zr; C₂₂H₁₈Cl₂N₄O₆Zr; C₂₆H₂₆Cl₂N₄O₆S₂Zr, C₂₆H₂₄Cl₄N₄O₆Zr, C₃₄H₂₆Cl₂N₄O₆Zr, C₂₂H₁₆Cl₄N₄O₆Zr, C₂₆H₂₄Cl₄N₄O₆Zr, C₃₄H₂₆Cl₂N₄O₆Zr, C₃₈H₂₈Cl₂N₆O₆Zr, Zr-glutamic acid-graphite oxide, and/or Zr-aspartic acid-graphite oxide.

Another and/or alternative non-limiting object of the present disclosure is the provision of a medical device that is formed of a base material and a zirconium containing coating, and the medical device includes tissue and/or polymer material that cannot be sterilized in high temperature environments (e.g., 38+° C.; 38+° C. to 100+° C. and all values and ranges therebetween; 38+° C. to 150+° C. and all values and ranges therebetween), and wherein the zirconium containing coating is applied to the tissue and/or polymer material to protect the tissue and/or polymer material from foreign materials and/or microbial growth, and which zirconium containing coating is removed from the tissue and/or polymer material prior to the medical device is implanted in the body of a patient.

Another and/or alternative non-limiting object of the present disclosure is the provision of a medical device that is formed of a base material and a zirconium containing coating, and the medical device includes tissue (e.g., leaflets, etc.) and/or polymer and/or fabric material (e.g., skirt, etc.) that cannot be sterilized in high temperature environments (e.g., 38+° C.; 38+° C. to 100+° C. and all values and ranges therebetween; 38+° C. to 150+° C. and all values and ranges therebetween), and wherein the zirconium containing coating is applied to the tissue and/or polymer and/or fabric material to protect the tissue and/or polymer and/or fabric material from foreign materials and/or microbial growth, and which zirconium containing coating is optionally removed from the tissue and/or polymer and/or fabric material prior to the medical device is implanted in the body of a patient, and wherein the medical device is a prosthetic heart valve.

Another and/or alternative non-limiting object of the present disclosure is the provision of a medical device that is formed of a base material and a zirconium containing coating, and the medical device includes tissue (e.g., leaflets, etc.) and/or polymer and/or fabric material (e.g., skirt, etc.) that cannot be sterilized in high temperature environments (e.g., 38+° C.; 38+° C. to 100+° C. and all values and ranges therebetween; 38+° C. to 150+° C. and all values and ranges therebetween), and wherein the zirconium containing coating is applied to the tissue and/or polymer and/or fabric material to protect the tissue and/or polymer and/or fabric material from foreign materials and/or microbial growth, and which zirconium containing coating is optionally removed from the tissue and/or polymer and/or fabric material prior to the medical device is implanted in the body of a patient, and wherein the medical device is a prosthetic heart valve, wherein the prosthetic heart valve includes an expandable metal frame, and wherein said expandable metal frame is optionally formed of a metal alloy that includes at least 15 atw. % rhenium.

Another and/or alternative non-limiting object of the present disclosure is the provision of a medical device is a prosthetic heart valve; and wherein the prosthetic heart valve includes an expandable frame, one or more leaflets, and one or more skirts; and wherein a zirconium containing coating is coated on an outer surface of one or more of the expandable frame, the one or more leaflets, and the one or more skirts. A non-limiting prosthetic heart valve that includes an expandable frame, one or more leaflets, and one or more skirts is disclosed in U.S. Ser. No. 18/400,781 filed Dec. 29, 2023, which is fully incorporated herein by reference. The zirconium containing coating can be partially (e.g., 0.1-99.999% of the outer surface and all values and ranges therebetween) or fully coated on the outer surface of the expandable frame, the one or more leaflets, and/or the one or more skirts.

These and other advantages will become apparent to those skilled in the art upon the reading and following of this description.

A more complete understanding of the articles/devices, processes and components disclosed herein can be obtained by reference to the accompanying drawings. The figure is merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawing and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall therebetween.

To aid the Patent Office and any readers of this application and any resulting patent in interpreting the claims appended hereto, applicants do not intend any of the appended claims or claim elements to invoke 35 U.S.C. 112(f) unless the words “means for” or “step for” are explicitly used in the particular claim.

What is claimed:

1. A medical device that includes a coating; said coating includes a) zirconium salt, b) zirconia, and/or c) Zr(IV) complexes.

2. The medical device as defined in claim 1, wherein said coating has a thickness of 0.01-500 μm.

3. The medical device as defined in claim 1, wherein said coating includes 30-100 wt. % of a) zirconium salt, b) zirconia, and/or c) Zr(IV) complexes.

4. The medical device as defined in claim 1, wherein said coating includes zirconium salt.

5. The medical device as defined in claim 4, wherein said zirconium salt includes one or more of zirconium nitrate, oxalic acid zirconium salt, AgZrPSi, AgZrP, zirconium phosphate-glycinediphosphonate and silver, zirconium phosphate-glycinediphosphonate and zinc, Ag_{0.2}H_{1.2}Zr_{1.9}(PO₄)₃·0.05H₂O, Ag_{0.1}H_{1.14}Zr_{1.94}(PO₄)₃·0.15H₂O, Ag_{0.2}Na_{0.4}(NH₄)_{0.84}Zr_{1.89}(PO₄)₃·0.3H₂O, Ag_{0.3}Na_{0.1}H_{1.04}Zr_{1.89}(PO₄)₃·0.2H₂O, Ag_{0.5}Na_{0.2}H_{0.3}(NH₄)_{0.32}Zr_{1.92}(PO₄)₃·0.2H₂O, and Ag_{0.4}K_{0.6}H_{0.36}Zr_{1.91}(PO₄)₃·0.1H₂O.

6. The medical device as defined in claim 1, wherein said antimicrobial coating includes zirconia.

7. The medical device as defined in claim 6, wherein said zirconia has a particle size of 0.001 μm to 0.5 μm.

8. The medical device as defined in claim 1, wherein said antimicrobial coating includes Zr(IV) complexes.

9. The medical device as defined in claim 8, wherein said Zr(IV) complexes are formed with amino acid or ligand.

10. The medical device as defined in claim 9, wherein said Zr(IV) complexes include one or more of C₂₀H₁₄Cl₂N₄O₆Zr; C₂₂H₁₈Cl₂N₄O₆Zr; C₂₆H₂₆Cl₂N₄O₆S₂Zr, C₂₆H₂₄Cl₄N₄O₆Zr, C₃₄H₂₆Cl₂N₄O₆Zr, C₂₂H₁₆Cl₄N₄O₆Zr, C₂₆H₂₄Cl₄N₄O₆Zr, C₃₄H₂₆Cl₂N₄O₆Zr, C₃₈H₂₈Cl₂N₆O₆Zr, Zr-glutamic acid-graphite oxide, and/or Zr-aspartic acid-graphite oxide.

11. The medical device as defined in claim 1, wherein said medical device includes tissue and/or polymer and/or fabric material that cannot be sterilized in high temperature environments.

12. The medical device as defined in claim 1, wherein said medical device is a prosthetic heart valve; said prosthetic heart valve includes an expandable frame, one or more leaflets, and one or more skirts; said coating is coated on an outer surface of one or more of said expandable frame, said one or more leaflets, and said one or more skirts.

13. The medical device as defined in claim 1, wherein at least a portion of said medical device is formed of a metal alloy that includes at least 15 atw. % rhenium.

14. The medical device as defined in claim 13, wherein metal alloy includes one or more alloying metals selected from the group consisting of aluminum, bismuth, chromium, cobalt, copper, hafnium, iridium, iron, magnesium, manganese, molybdenum, nickel, niobium, osmium, platinum, rhodium, ruthenium, silicon, silver, tantalum, technetium, tin, titanium, tungsten, vanadium, yttrium, and zirconium; said metal alloy has a) an increase of at least 10% in ductility as compared to said metal alloy that is absent rhenium, and/or b) an increase of at least 10% in tensile strength.

15. The medical device as defined in claim 1, wherein said coating is applied to said medical device at a concentration of at least 5.7 μg/mL.

16. The medical device as defined in claim 15, wherein said coating is applied to said medical device at a concentration of 5.7 μg/mL to 200 μg/mL.

17. A medical device that includes a removable antimicrobial coating; said medical device is a prosthetic heart valve; said prosthetic heart valve includes an expandable frame and one or more a) a leaflet and b) a skirt; said coating includes a) zirconium salt, b) zirconia, and/or c) Zr(IV) complexes; said coating is coated on an outer surface of said leaflet and/or said skirt; said coating has a thickness of 0.01-500 μm.

18. A method of inhibiting microbial growth on a medical device and/or sterilizing a medical device comprising:
a. providing a medical device; and
b. applying an antimicrobial coating on at least a portion of said medical device; said coating including a) zirconium salt, b) zirconia, and/or c) Zr(IV) complexes.

19. The method as defined in claim 18, further including the step of at least partially removing said coating from said medical device prior to inserting said medical device into a patient.

20. A method of inhibiting microbial growth on a medical device and/or sterilizing a medical device comprising:
a. providing a medical device; said medical device is a prosthetic heart valve; said prosthetic heart valve includes an expandable frame and one or more a) a leaflet and b) a skirt;
b. applying an antimicrobial coating on at least a portion of said medical device; said coating including a) zirconium salt, b) zirconia, and/or c) Zr(IV) complexes; said coating is coated on an outer surface of said leaflet and/or said skirt to inhibit or prevent microbial growth on said outer surface of said leaflet and/or skirt that includes said coating; said coating has a thickness of 0.01-500 μm; and
c. at least partially removing said coating from said medical device prior to inserting said medical device into a patient.
